(19) United States
(12) Patent Application Publication
(10) Pub. No.: US 2023/0390770 A1
(43) Pub. Date: Dec. 7, 2023

(54) High Throughput Microfluidics for Analysis of Immune Cell Activation

(71) Applicant: The General Hospital Corporation, Boston, MA (US)

(72) Inventors: Rohan Thakur, Cambridge, MA (US); James Malcolm Heather, Somerville, MA (US); Shannon Leigh Stott, Winchester, MA (US)

(21) Appl. No.: 18/035,888

(22) PCT Filed: Nov. 9, 2021
(86) PCT No.: PCT/US2021/058665

§ 371 Date: May 8, 2023

Related U.S. Application Data
(60) Provisional application No. 63/111,131, filed on Nov. 9, 2020.

(51) Int. Cl. B01L 3/00; C12Q 1/6874

(52) U.S. Cl. CPC B01L 3/502784; B01L 3/50273; C12Q 1/6874; B01L 2200/0647; B01L 2300/021; B01L 2300/0645; B01L 2200/0636

(57) ABSTRACT

Provided herein are microfluidic platforms and methods of use thereof for generating, tracking, monitoring, and analyzing thousands of droplets per second for interactions between two or more particles, such as cells, encapsulated in individual droplets, wherein the individual droplets are uniquely identified by specific ratios of multiple different optical barcodes and at least one sequence barcode per droplet.

### CLAIM OF PRIORITY

This application claims the benefit of U.S. Provisional Patent Application No. 63/111,131, filed on Nov. 9, 2020, which is incorporated by reference herein in its entirety.

### FIELD OF THE INVENTION

The present disclosure relates to detecting interactions between interacting partners, such as individual cells.

### BACKGROUND

Cellular immunotherapies show enormous potential in addressing clinical oncological need. By engineering T-cell receptors (TCRs), immunotherapies could be developed that specifically elicit an immune response, e.g., to a patient's tumor. However, the number of possible sequences to screen is enormous and therefore there is a pressing need for tools that can rapidly assess T-cell activation. Existing tools generally have a low throughput, lack single cell resolution, and/or lack the ability to investigate cell pairs (e.g., antigen presenting cells (APCs) and T-cells or other TCR-expressing cells). The rapidly expanding field of immuno-oncology has transformed cancer patient care, yet it remains challenging to screen and identify the most promising immunotherapeutic options.

Cell-based therapeutics like adoptive T-cell transfer have shown encouraging results in clinical trials to confer protective immunity, but require a complicated series of steps including activation, clonal expansion, differentiation, and migration to tissue sites. T-cell recognition of peptides displayed by antigen presenting cells (APC) also forms the basis of immune rejection in immuno-oncology. To minimize treatment-induced adverse reactions while maximizing treatment efficacy, T-cell responses must specifically target antigens via T-cell receptors.

Prior devices using droplet microfluidics are known in the art. Some devices have clinical applications in cancer. However, high-throughput isolation and detection of either the T-cell receptors (TCRs) or cognate antigens that drive immune responses remains extremely challenging, and capturing functionally interacting TCR-APC pairs is harder still.

It is therefore of interest to develop new devices and methods that can profile T-cell activation in response to a presented antigen, e.g., a cancer neoantigen, and correlate the activation profile to specific TCR sequences.

### SUMMARY

The present disclosure is based, at least in part, on the concept that if one combines optical barcodes, e.g., fluorescent barcodes, and standard sequencing barcodes, such as Unique Molecular Identifiers (UMIs), with ordered pairs of particles, such as cells, encapsulated together in individual droplets, in a microfluidic platform that provides controlled flow, sorting, and selection of droplets that contain activated particles, optical tracking, recording, and analysis, then one can produce, monitor, and analyze many thousands of droplets per second for interactions between two particles, e.g., cells, within individual droplets, e.g., T-cell activation in response to an antigen, e.g., presented on the surface of an APC, and then correlate activation profiles with specific genetic sequence information of the interacting cells.

In one aspect, the present disclosure provides microfluidic platforms that can profile T-cell activation in response to a presented antigen, such as a cancer neoantigen, and correlate the activation profile to specific TCR sequences using a dual barcoding approach. The platforms also include monitoring of T-cell activation via an activation signal, and records an activation profile correlated to unique barcode identifiers in each droplet.

In one aspect, the disclosure provides methods for analyzing an interaction between two or more particles. The methods include or consist of (a) inertially ordering the particles into spaced and ordered streams of particles; (b) co-encapsulating in individual droplets two or more of the spaced and ordered particles and an activation reporter to form a plurality of target droplets; (c) co-encapsulating in individual droplets a plurality of different optical barcodes to generate a plurality of barcoded droplets, wherein a specific ratio of the different optical barcodes is used to uniquely identify each of the individual barcoded droplets; (d) determining interaction between the particles in each target droplet by monitoring each target droplet for a presence or absence of the activation reporter to identify target droplets positive for interacting, activated particles; (e) merging each identified target droplet with an adjacent barcoded droplet to generate merged droplets; and (f) sequencing nucleic acids in the merged droplets to determine the sequence of any nucleic acids in the particles and to determine the sequence of any barcodes in the merged droplets; wherein step (c) further includes co-encapsulating in the individual barcoded droplets at least one sequence barcode to generate a plurality of dual barcoded droplets and step (e) includes merging each identified target droplet with an adjacent dual barcoded droplet; or wherein steps (b) and (c) are combined to co-encapsulate in individual target droplets the two or more particles and the activation reporter as well as the plurality of different optical barcodes to generate the target droplets, and step (e) includes merging each identified target droplet with a second droplet including a sequence barcode to form the merged droplet; or wherein step (c) further includes merging the target droplets with the barcoded droplets to generate optically barcoded target droplets, and step (e) includes merging each identified optically barcoded target droplet with a second droplet comprising a sequence barcode.

In these methods, the particles can be T-cells and Antigen Presenting Cells (APCs) from a patient who has cancer, and method can be used to analyze TCR-antigen interactions in a sample of a patient's tumor and information about the patient's TCR-antigen interactions is used to select a TCR-based immunotherapy that will recognize the patient's tumor to stimulate an anti-tumor response. In other embodiments, the particles can be immune cells and diseased cells from a patient who has an autoimmune disease, and the method can be used to analyze interactions in a sample between the patient's immune cells and diseased cells to identify immune cells responsible for an unwanted autoimmune response.

In another embodiment, the particles can be tumor cells from a patient and one or more specific drugs taken by the patient whose tumor has developed a resistance to the one or more specific drugs, and the method can be used to analyze interactions between the patient's cells and the one or more specific drugs to identify genetic mutations responsible for the patient's drug resistance.

In other embodiments, the particles are a bacteria or a virus that has become resistant to a drug, and the method can be used to determine any genetic cause of the drug resistance.

In certain embodiments, inertially ordering the particles includes flowing the particles through one or more channels at a flow rate that is controlled to induce inertial focusing. For example, the one or more channels can include or consist of one or more curved channels having a Dean number of up to about 30. In some embodiments, the curved channel is symmetrically curved and a channel Reynolds number (Rc) of between about 0.5 and 5.0 causes focusing of particles into two longitudinally ordered streams of particles. In other embodiments, the curved channel is asymmetrically curved and a channel Reynolds number (Rc) of between about 1.0 and 15.0 causes focusing of particles into a single longitudinally ordered stream of particles.

In some embodiments, the curved channel is asymmetrically curved and a mean channel velocity (Re) is set to about 2.5 to 5.0, wherein Re equals ⅔ of the channel Reynolds number (c). In some embodiments, the Dean number for the curved channel ranges from about 1 to about 20, and wherein a ratio of particle size to hydraulic diameter of the first microchannel is less than about 0.5.

In various embodiments, each optical barcode in the plurality of optical barcodes includes an injector ID nucleic acid sequence, a fluorescent molecule, and a unique molecular identifier (UMI), wherein all injector ID nucleic acid sequences for one fluorescent color are the same, but are different from injector ID nucleic acid sequences for optical barcodes having a different fluorescent color, and wherein all UMIs are different.

In some embodiments, the sequence barcode includes or is a UMI.

In various embodiments, the optical barcodes confer one or more optical properties selected from the group consisting of an absorbance, a birefringence, a color, a fluorescence characteristic, a luminosity, a photosensitivity, a reflectivity, a refractive index, a scattering, or a transmittance of the particle, or a component thereof.

In some embodiments, the target droplets and the dual barcoded droplets are merged by applying an electric field that causes destabilization of the droplets such that they are merged together.

In certain embodiments, the target droplets and the dual barcoded droplets are merged by droplet-stream merger, droplet-jet merger, or both.

In various embodiments, the particles are selected from the group consisting of cells, eggs, bacteria, fungi, virus, algae, any prokaryotic or eukaryotic cells, organelles, exosomes, beads, reagents, drugs, small molecules, proteins, antibodies, enzymes, and nucleic acids..

In some embodiments, the particles include or are a T-cell and an antigen presenting cell (APC) and the activation reporter is a calcium activation reporter.

In certain embodiments, the sequencing includes single-cell RNA sequencing or single-cell DNA sequencing. In differing embodiments of the methods, one or more of the steps are performed in a microfluidic device.

In various embodiments, the methods are carried out at a flow rate that enables production and monitoring of at least 100, 500, 1000, 2500, 5000, 7500, or 10,000 droplets per second.

In another aspect, the present disclosure provides microfluidic systems that include: (a) a dual barcoded droplet preparation module including a plurality of channels for receiving one or more barcodes and a droplet generator including a nozzle in fluid communication with the plurality of channels; (b) an inertially ordered cell encapsulation module including (i) a microchannel having an inlet, an outlet, and a minimum cross-sectional dimension D configured to receive a fluid sample containing multiple particles having a maximum individual cross-sectional dimension of at least 0.1 D, and (ii) a droplet generator including a nozzle in fluid communication with the outlet of the microfluidic channel; (c) an incubation and activation profiling module having an inlet end, a middle section, and on outlet end, wherein the inlet end includes a central channel for droplets and a plurality of microchannels arranged in fluid communication with and one or both sides of the central channel to allow excess fluid and other waste materials in the fluid sample to flow out of the central channel while maintaining droplets within the central channel, and wherein the outlet end includes a narrowing channel to allow the droplets to become arranged in single file when exiting the outlet end; (d) an optical barcode detection module including one or more optical detection devices; and (e) a selective droplet merging module including a channel and an electrode configured to apply an electric field in the channel sufficient to cause adjacent droplets to merge into one larger droplet.

In different embodiments, the systems can further include one or more pumping mechanisms in fluid communication with the microfluidic system and arranged to move a fluid sample through the microfluidic system.

In some embodiments, the electrode is controlled to apply an electric field when the one or more optical detection devices signal an activated particle.

In certain embodiments, the microchannels of the systems include a curved microchannel and has a Dean number of up to about 30. In some embodiments, the curved microchannel is symmetrically curved and has a channel Reynolds number (Rc) of between about 0.5 and 5.0 to cause focusing of particles into two longitudinally ordered streams of particles. In some embodiments, the curved microchannel is asymmetrically curved and has a channel Reynolds number (Rc) of between about 1.0 and 15.0 to cause focusing of particles into a single longitudinally ordered stream of particles. In other embodiments, the curved microchannel is asymmetrically curved and has a mean channel velocity (Re) set to about 2.5 to 5.0, wherein Re equals ⅔ of a channel Reynolds number (Rc). In certain embodiments, the Dean number for the curved microchannel ranges from about 1 to about 20, and wherein a ratio of particle size to hydraulic diameter of the first microchannel is less than about 0.5.

In some embodiments, the microfluidic systems further include one or more controllers having hardware or software, or both hardware and software, configured to control one or more of: (i) the one or more pumping mechanisms to regulate the flow rate of the fluid sample within the microfluidic system, (ii) the injection of different optical barcodes into the dual barcoded droplet preparation module in precise, unique ratios per droplet, (iii) the one or more optical detection devices to detect and/or monitor for an activation reporter in any activated target droplets, (iv) the one or more optical detection devices to detect and/or monitor optical barcode ratios per droplet, and (iv) the electrode.

In certain embodiments, the microfluidic systems further include one or more conduits arranged to flow a fluid sample containing droplets between the dual barcoded droplet preparation module and the inertially ordered cell encapsulation module; between the inertially ordered cell encapsulation module and the incubation and activation profiling module; between the incubation and activation profiling module and the optical barcode detection module; and/or between the optical barcode detection module and the selective droplet merging module.

In some embodiments, the microfluidic systems further comprising a sequencing system such as a sequencing-by-synthesis system.

As used herein, unless otherwise indicated, the terms “barcode” or “barcode sequence” refer to any unique sequence label (e.g., nucleic acid and/or protein) that can be coupled to at least one nucleotide sequence for later identification of the at least one nucleotide sequence.

As used herein, unless otherwise indicated, the terms “oligonucleotide barcode sequence” or “oligonucleotide barcode” refer to an oligonucleotide (e.g., DNA or RNA) having a sequence which can identify and/or distinguish one or more target molecules to which it is attached. Oligonucleotide barcode sequences are typically short, e.g., about 5 to 20 bases in length, and they can be attached to one or more target molecules or amplification products thereof. Oligonucleotide barcode sequences may be single or double stranded.

As used herein, unless otherwise indicated, the term “optical barcode” refers to a molecule that produces an optically detectable signal that can identify and/or distinguish one or more target molecules or cells with which they are co-encapsulated. Optical barcodes include a reporter molecule, such as a fluorescent molecule, attached to an oligonucleotide or polypeptide barcode. In general, the optical barcode does not hybridize with the sequence barcode. Instead, the optical barcode can be correlated with the sequence barcode via encapsulation in the same droplet and without hybridization to the sequence barcode.

As used herein, unless otherwise indicated, the term “unique molecular identifier (UMI)” or “UMI” refers to a type of oligonucleotide barcode having a sequence that can be used to identify and/or distinguish one or more target molecules to which the UMI is attached. UMIs are typically short, e.g., about 5 to 20 bases in length, and may be attached to one or more target molecules or amplification products thereof. UMIs may be single or double stranded. In some embodiments, both a nucleic acid barcode sequence and a UMI are incorporated into a nucleic acid target molecule or an amplification product thereof. Generally, a UMI is used to distinguish between molecules of a similar type within a population or group, whereas an oligonucleotide barcode sequence is used to distinguish between populations or groups of molecules. In some embodiments, where both a UMI and an oligonucleotide barcode are utilized, the UMI is shorter in sequence length than the nucleic acid barcode sequence. In some embodiments, where both a UMI and an oligonucleotide barcode sequence are used, the UMI is incorporated into the target nucleic acid or an amplification product thereof prior to the incorporation of the oligonucleotide barcode sequence. In some embodiments, where both a UMI and an oligonucleotide barcode sequence are used, the oligonucleotide barcode sequence is incorporated into the UMI or an amplification product thereof subsequent to the incorporation of the UMI into the target nucleic acid or an amplification product thereof.

As used herein, unless otherwise indicated, the term “droplet” refers to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g., an aqueous phase fluid (e.g., water). In some embodiments, the second fluid phase will be an immiscible phase carrier fluid. Thus, droplets according to the present disclosure may be provided as aqueous-in-oil emulsions or oil-in-aqueous emulsions. Droplets may be various sizes and/or shapes. For example, droplets can generally range from 1 μm to 1000 μm in diameter. In some examples, droplets can be sphere-shaped or oblong-shaped. In some examples, droplets can have a volume ranging from 1 fL to 1 nL. Droplets can be used to encapsulate cells, extracellular vesicles, viruses, nucleic acids (e.g., DNA), enzymes, reagents, drugs, and/or other particles. The term droplet may be used to refer to a droplet produced in, on, or by a microfluidic device and/or flowed from or applied by a microfluidic device.

As used herein, unless otherwise indicated, the term “dual barcoded droplet” refers to a droplet comprising one or more optical barcodes and one or more oligonucleotide barcodes.

As used herein, unless otherwise indicated, the term “particle” refers to a small discrete mass of solid or liquid matter, such as a cell or a solid particle that can be discretely transported in a fluid stream. Particles can be any size which allows them to be transported within a microfluidic channel. For example, particles can have an average hydrodynamic size that is between 1 μm to 100 μm. The particle size is limited only by channel geometry; accordingly, particles that are larger and smaller than the above-described particles can be used. Non-limiting examples of particles include cells, eggs, bacteria, fungi, virus, algae, any prokaryotic or eukaryotic cells, organelles, exosomes, beads, reagents, drugs, small molecules, proteins, antibodies, enzymes, and nucleic acids.

As used herein, unless otherwise indicated, the term “fluid” refers to a gas or liquid, such a liquid biological sample (e.g., whole blood).

The simultaneous profiling of cell interactions of pairs of cells, such as T-cell activation by APCs, and cell sequencing provided by the present disclosure has not heretofore been demonstrated for pairs of interacting cells. The massive throughput of the workflow of the devices described herein enable processing thousands of cell pairs, in up to about 10,000 droplets per second, which enables extremely rapid exploration of the TCR sequence space. The platforms described herein can be employed, for example, to identify new cancer neoantigens and the TCRs that can recognize them. The platforms can also be incorporated into immunotherapeutic pipelines and can enable the rapid development of personalized cellular immunotherapies and cancer vaccines and provide functional phenotyping and sequencing at the single cell level.

The T-cell Activation Profiling and Sequencing (TAP-Seq) platform technology described herein bridges the gap between low throughput functional studies of T-cell activation and high throughput single cell RNA-sequencing. The dual barcoding approach described herein enables mapping fluorescence time series data with the transcriptome of the associated cells. More broadly, this technology enables researchers to elucidate the complex interplay between phenotype and transcriptome of interacting cells.

The TAP-Seq platform described herein enables the functional annotation of TCR and antigen libraries at the single cell level. Because the platforms are built upon ultra high-throughput microfluidic modules, thousands of cell pairs per second can be investigated. Furthermore, the platforms enrich for activated cell pairs and can potentially process large libraries with incredibly rare immunogenic pairs. Therefore, it is feasible to rapidly explore the vast TCR sequence space to optimize tumor antigen recognition. Additionally, the platforms can be used as flexible and quantitative tools for personalized immunotherapies including cancer vaccines and CAR-T pipelines.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, scientific articles, published patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a high level system diagram of the different modules of the T-cell Activation Profiling and Sequencing (TAP-Seq) platform described herein.

FIG. 2A is a schematic diagram illustrating a dual barcoded (optical barcode and oligonucleotide sequence barcode) droplet preparation module. Droplets are generated through the co-encapsulation of barcoded beads with a unique combination of fluorescent barcode molecules and DNA oligonucleotide barcodes providing each droplet with a unique combination of these two different types of barcodes. This enables identification both optically and in the sequence space.

FIG. 2B is a schematic diagram illustrating an inertially ordered cell encapsulation module. In parallel, individual target particles, e.g., T-cells and antigen presenting cells (APCs), are co-encapsulated into droplets. To overcome Poisson distribution limitations, the target particles, e.g., cells, are inertially ordered prior to generating the two-cell pairs. Included in each droplet containing a unique two-cell pair is a reporter, e.g., an optical reporter, such as a fluorescent reporter, to signal cell activation, e.g., calcium mobilization—a hallmark of T-cell activation.

FIG. 2C is a schematic diagram illustrating an incubation and activation profiling module showing cell activation signaling, e.g., calcium signaling, over time in an inset. Target particles, e.g., cell pairs are incubated while the T-cell activation is recorded.

FIG. 2D is a schematic diagram illustrating an optical barcode detection module showing the ratios of three different fluorescently labeled optical barcodes. Activated cell pairs are paired with the dual barcoded droplets, to link the detected optical code with each cell pair.

FIG. 2E is a schematic diagram illustrating a selective droplet merging module including an electrode that is used to selectively and temporarily disrupt adjacent droplets causing them to merge into one droplet that contains two cells, three different optical barcodes (red, yellow, and blue in this embodiment), and a unique sequencing barcode (UMI), in or attached to the gray bead within the larger droplet. To both reduce costs and prioritize sequencing depth, in this embodiment, only droplets that contain activated target particles, e.g., cell pairs, as identified optically, e.g., fluorescently, are selectively merged with the dual barcoded droplets. Merged droplets are then sequenced, and, in this embodiment, capturing the identity of the peptide antigen, the TCR sequence, and the encapsulated DNA barcodes.

FIG. 2F is a diagram illustrating the correlation of optical barcodes based on color and fluorescence intensity with cell activation profiles (e.g., calcium signaling over time) with DNA barcode (UMI) information and cell sequencing results. The correlation between the optical (fluorescent) barcodes and the sequencing (UMI) barcodes is thus linked to the observed T-cell activation profile to the TCR and peptide antigen sequence.

FIG. 2G is a diagram illustrating the mapping of activation profiles (e.g., calcium signaling over time) of cells in individual droplets in small droplet clusters to transcriptome data and tracked to particular cells located in individual droplets. Activation profiles can be included in computational clustering pipelines alongside transcriptional profiles.

FIG. 3A is a schematic diagram illustrating the use of inertial microfluidics for cell ordering and encapsulation while simultaneously adding three different fluorescently labeled barcodes (optical barcodes), and then packing of the droplets into a holding (incubation) chamber where the individual droplets are densely packed as they move through the chamber, and are tracked, e.g., microscopically, and any cell activation is recorded and linked to the individual droplets that show activation, so that this information follows the individual droplet within the system processor and memory.

FIG. 3B is a microscope image that shows inertial ordering and longitudinal spacing of cells prior to encapsulation and then encapsulation of individual cells into individual droplets. Thereafter, pairs of droplets can be merged such that each, or most, of the merged droplets contain two cells. The cells flowing along the top of the microfluidic channel may be a first type of cell, and the cells flowing along the bottom of the microfluidic channel may be a second type of cell, so that the two types of cells alternate as they are encapsulated.

FIG. 3C is a schematic diagram that represents a single droplet that contains both an activated T-cell with receptor (TCR) in the lower left of the droplet and an antigen presenting cell (APC) in the upper right of the droplet, wherein cell activation is represented by the “rays” emanating from the TCR in the lower left.

FIG. 3D is a microscope image of fluorescently labeled (optically barcoded) droplets that have different levels of label to distinguish different droplets, each unique, and one droplet of the group shows a cell within a droplet that has been activated, as signaled by a separate fluorescent emission.

FIG. 3E is a schematic diagram that shows a workflow of incubating a large number of uniquely labeled droplets for a sufficient time to permit cell activation, and then adding to these droplets, ordered in single file, a second series of droplets that are each uniquely labeled with UMIs, so that the optically labelled and the UMI labeled droplets are arranged in alternating order, with one of each type of droplet arranged in adjacent pairs of droplets, with a controlled spacing between these pairs of droplets. Thereafter, the fluorescent (e.g., color) information in the optically barcoded droplets is read, and then they are merged with the adjacent UMI barcoded droplets to generate dual barcoded droplets, each with a unique fluorescent optical barcode and a unique UMI barcode.

FIG. 3F is a microscope image of the reinjection and pairing of the two types of barcoded droplets.

FIG. 3G is a microscope image of the selective merging of adjacent droplets that include an activation signal. The image shows the adjacent droplets traveling in a channel perpendicular to the selective merger electrode. Droplet merger is triggered on only droplets of interest by linking detection of the activation signal to merger electrode activation.

FIG. 4 is a schematic diagram showing the inertial ordering of particles flowing through a straight microfluidic channel.

FIG. 5 is a schematic diagram showing the separation, ordering, and focusing of particles in a fluid passing through a symmetrically curved serpentine microfluidic channel.

FIG. 6A is a schematic diagram of a curved portion of a microfluidic flow channel of the inertial cell ordering channel, as shown in FIG. 5, in the inertially ordered cell encapsulation module. These microfluidic channels are designed to exploit the properties of Dean flow and Reynolds numbers to use the resulting forces to position cells precisely within the channel, with adjustable longitudinal distances between each cell. The resulting flow streams of cells enter a series of asymmetric curved channels and emerge as a single line of individual cells traveling within a define streamline position within the channel. By incorporating inertial ordering into the platform design, Poisson distribution limits typically seen in droplet generation technologies are overcome, which allows for more efficient cell pairing with minimal cell loss.

FIG. 6B is a flow and circulation model of the internal fluid flows through a cross-section of the inertial cell ordering channel of FIG. 6A, taken from Martel and Toner, Phys. Fluids, 24(3):32001 (2012).

FIG. 6C is a fluid mechanics equation and diagram showing the forces on a single cell passing through the inertial cell ordering channel of FIG. 6A, taken from Martel and Toner, Annu. Rev. Biomed. Eng. 2014. 16:371-96.

FIG. 6D is a microscope image of cells being ordered within an inertial cell ordering channel of FIG. 6A.

FIG. 7 is a schematic diagram showing a portion of the droplet incubation and activation profiling module containing packed droplets, with enlarged insets of an individual droplet at the top left of the figure showing a pair of cells that did not bind and caused no cell activation (and the related graph inset showing no calcium signaling), and a second droplet in the lower right of the figure showing bound cells and cell activation (and the related graph inset showing calcium signaling over time).

FIG. 8A is a series of microscope images of three sections of a droplet incubation and activation profiling module (inlet end, middle, outlet end) in which droplets are collected and moved from the left inlet end of the module in the figure through an initial sieving section, which removes excess fluid, through a middle section in which the droplets are more tightly packed and ordered, while still moving and being optically scanned for cell activation information, and then moving into a triangular single file ordering outlet end section, to pass into a microfluidic channel in single file, with cell activation profile information recorded and tracked to each individual droplet.

FIG. 8B is a series of still microscope video images over time (from 0 to 300 μs showing selective merging of a droplet comprising a bead (arrow at 0 μs) with a larger droplet at a frequency of 3000 Hz. In the final panel, at 300 μs, the bead is merged with the larger droplet.

FIG. 8C is a microscope image showing when a droplet comprising a bead begins to merge with the larger droplet directly adjacent to an electrode.

FIG. 8D is a microscope image of a middle section of the droplet incubation and activation profiling module of FIG. 8A containing hundreds of tightly packed droplets with three droplets showing a fluorescent signal (marked with an arrow) indicating cell activation (scale bar is 1000 microns).

FIG. 9 is a schematic illustrating processing of patient derived TCR-antigen pairs on the TAP-Seq platform. A biopsy sample collected from a cancer patient is conventionally analyzed by tissue staining to obtain information regarding the types of cells present in the tumor, whereas methods and devices described herein enable identification of the sequence of immunogenic tumor antigens within the tumor.

### DETAILED DESCRIPTION

The microfluidic TAP-Seq platform described herein is built upon droplet microfluidic principles, which manipulate discrete, miniature volumes of fluids in immiscible fluid phases within laminar flow regimes. By controlling the channel geometry, it is possible to create highly uniform droplets at rates of several hundred to tens of thousands per second. Each droplet can function as a microscale bioreactor, enabling both incredible throughput and multiplexed analysis of biological samples. Following droplet formation, precise manipulation via electric forces enable droplet merging, injection, and sorting to coordinate biochemical reactions and enrich for rare subpopulations.

The TAP-Seq platform can be used to analyze any interaction of interest by encapsulating a pair of molecules, e.g., cells, that potentially interact in one droplet and encapsulating dual barcodes in another droplet. An activation reporter is also encapsulated in the droplet with the pair of molecules, e.g., cells, to provide a detectable signal if and when the pair of molecules interact so that the droplet can be tracked as the droplet moves through the device. This detectable signal can be used to distinguish droplets in which the pair of molecules interact from droplets in which no interaction is taking place. A droplet identified as including interacting molecules, e.g., cells, is then selectively merged with a droplet including dual barcodes, which allows the barcode sequences to be uniquely associated with the cell so that all sequencing reads containing the barcode are known to originate from the cell.

It should be understood that the TAP-Seq platform can be used to analyze interactions between multiple molecules by encapsulating multiple molecules in a droplet. Any number of molecules can be encapsulated in a droplet and analyzed as described herein to determine whether the molecules interact. In some examples, multiple molecules (e.g., three, four, five, six, or more molecules) can be encapsulated in a droplet as long as each type of molecule pair has its own reporter molecule specific for a targeted interaction.

Any type(s) of molecules (particles) can be encapsulated in a droplet and analyzed as described herein to determine whether the molecules interact. For example, when encapsulating a pair of molecules in a droplet, non-limiting examples of a pair of molecules includes a pair of cells (e.g., T-cell and APC, T-cell and B-cell, T-cell and dendritic cell), a cell and an exosome, a cell and a protein (e.g., a cell and an antibody), a cell and a nucleic acid (e.g., a cell and a therapeutic oligonucleotide), a cell and an extracellular vesicle, a cell and a drug, a cell and a pathogen (e.g., a cell and a virus, a cell and a bacteria), a drug and a pathogen, a protein and a drug, a nucleic acid and a drug, and a protein and a nucleic acid.

In another example, when encapsulating multiple molecules in a droplet, non-limiting examples of multiple molecules include a pair of cells and a drug, a pair of cells and a protein, a pair of cells and a nucleic acid, or a pair of cells and a pathogen. In some examples, multiple molecules in a droplet include any combination of one or more cells, one or more proteins, one or more nucleic acids, one or more drugs, and one or more pathogens.

Accordingly, the TAP-Seq platform can be used to analyze a wide range of interactions that can be useful for various clinical purposes including, but not limited to, identifying patients suitable for a particular treatment, assessing efficacy of a treatment, monitoring progress of a disease in a patient, and predicting relapse of a disease in a patient.

For example, when the patient has cancer, the TAP-Seq platform can be used to analyze TCR-antigen interactions in the patient's tumor and information about the patient's TCR-antigen interactions can be used to select a TCR-based immunotherapy that will recognize the patient's tumor to stimulate an anti-tumor response.

In another example, when the patient has an autoimmune disease, the TAP-Seq platform can be used to analyze interactions between the patient's immune cells and other cells from the patient to identify immune cells responsible for the unwanted autoimmune response.

In another example, when the patient has developed resistance to a treatment, the TAP-Seq platform can be used to analyze interactions between the patient's cells and a drug to identify genetic mutations responsible for the patient's drug resistance. Similarly, drug resistant pathogens (e.g., bacteria, viruses) can be analyzed to identify genetic causes of drug resistance.

Immunotherapies have great promise in directing a cancer patient's immune system to attack their tumor. However, a critical step in the realization of these therapies is the functional evaluation of T-cell activation against cancer associated antigens. The T-cell activation profile not only indicates recognition of an antigen, but also the strength and specific T-cell effector function in response to the antigen. The microfluidic TAP-Seq platform disclosed herein can link optical phenotypes with the transcriptome of cells, giving unprecedented functional information at a single cell level. The TAP-Seq platform can be used to quantitatively profile immune cell activation of synthetic TCR-APC pairs.

First, TCR and antigen sequences with well-characterized immunogenicity are selected. By comparing these single cell activation profiles with bulk activation profiles, we can verify that TAP-Seq reproduces the immune synapse formed in traditional in vitro systems and that T-cell transcriptional changes are well correlated with the measured activation dynamics. The platform can distinguish cognate and mismatched TCR- antigen pairs via the measured activation dynamics. By sequencing the tumor cells and lymphocyte infiltrate from a patient's solid biopsy, a list of potential cancer neoantigens and tumor specific TCR sequences is created. Then a dual library of synthetic T-cells and APCs representing the potential interacting cell pairs in a tumor is created. Using the TAP-Seq methodology, this library is then screened for activated T-cells, identifying productive TCR-APC pairs specific to the patient's tumor. T cell recognition of cancer neoantigens forms the basis for many promising immunotherapies. However, existing technologies are insufficient in measuring T-cell activation in response to the presented neoantigens.

The TAP-Seq platform addresses these issues by:
Ultrahigh throughput (thousands of droplets per second) functional analysis of T-cell & APC cell pairs.
Fully autonomous microfluidic barcoding, incubation, and sorting workflow via custom control software.
Controlled manipulation and pairing of individual T-cells with individual APCs
Continuous fluorescence measurement that enable the profiling of T-cell activation as a function of time.
Computational mapping of activation profile to T-cell sequence via novel dual barcoding approach.
Extensive applications in cancer, including integrated multi-omic sequencing and study of cell-cell interaction, with potential for immediate impact in CAR-T development.

### Tap-Seq Platform Overview

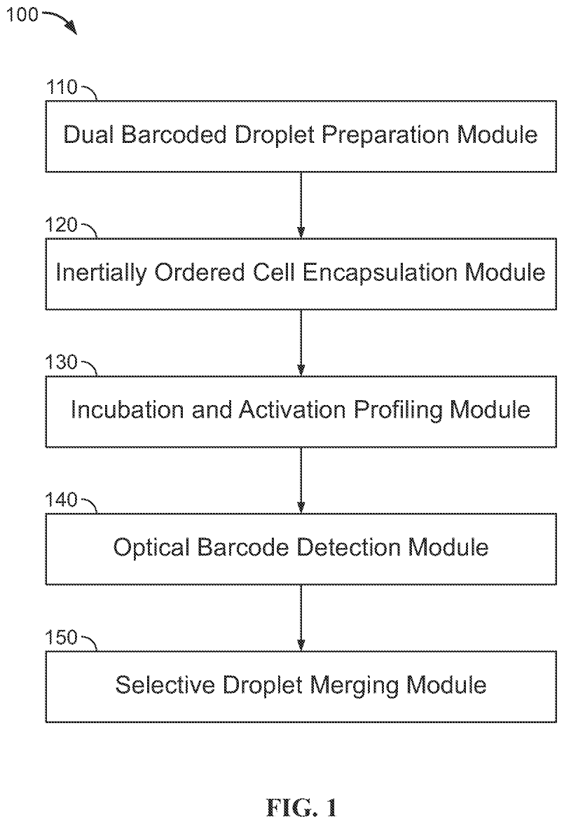

FIG. 1 provides a high-level schematic of the different modules that are combined into the TAP-Seq platform. As shown in FIG. 1, the TAP-Seq platform includes a dual barcoded droplet preparation module 110, an inertially ordered cell encapsulation module 120, incubation and activation profiling module 130, optical barcode detection module 140, and a selective droplet merging module 150.

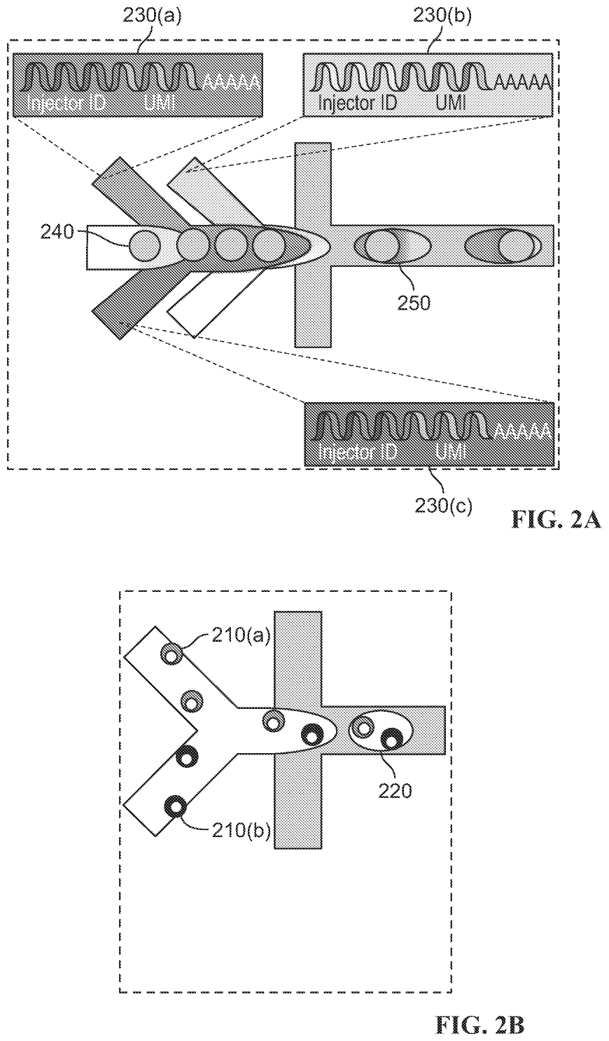

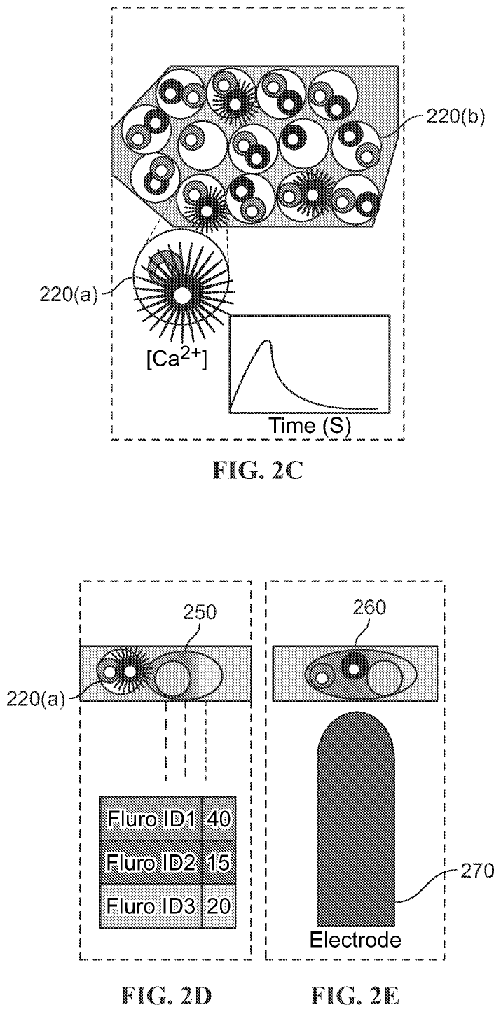

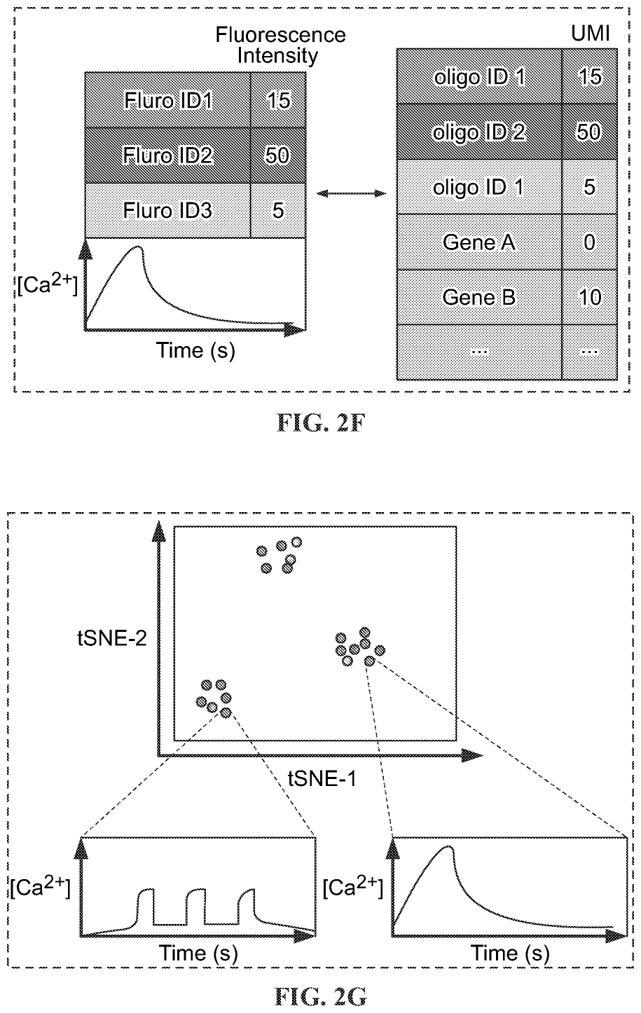

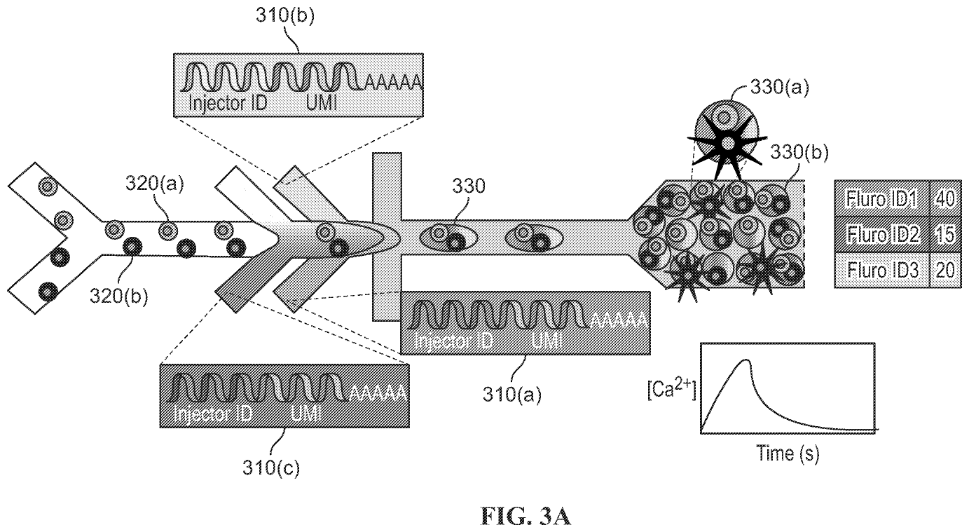

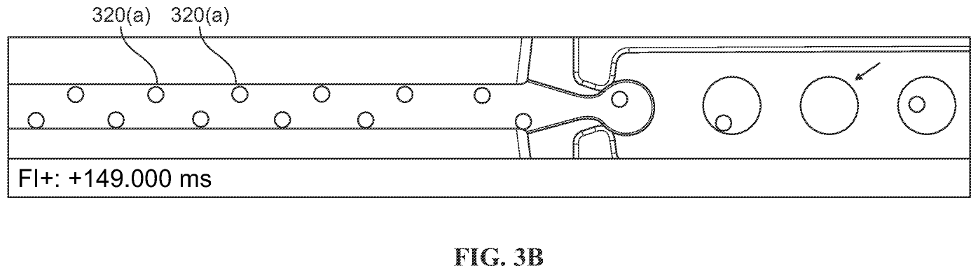

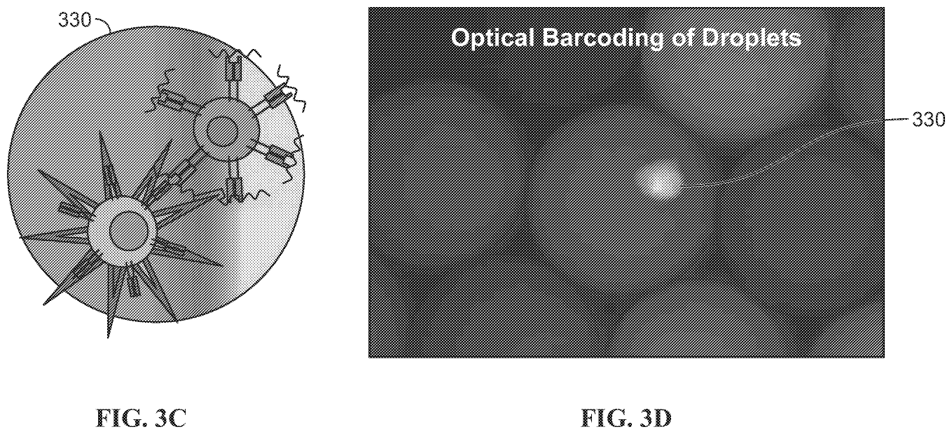

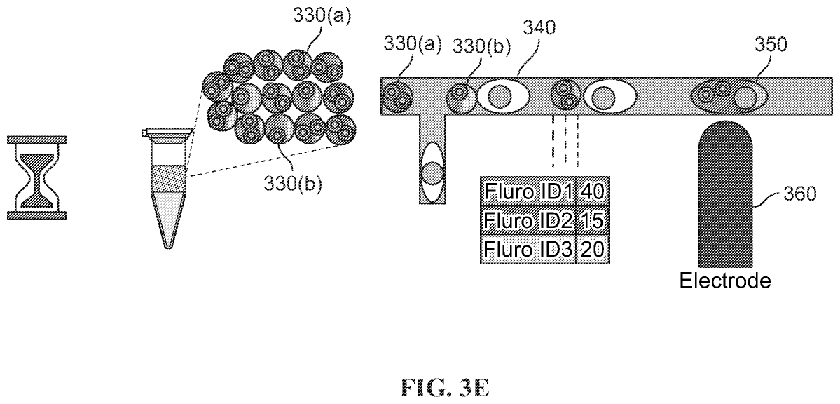

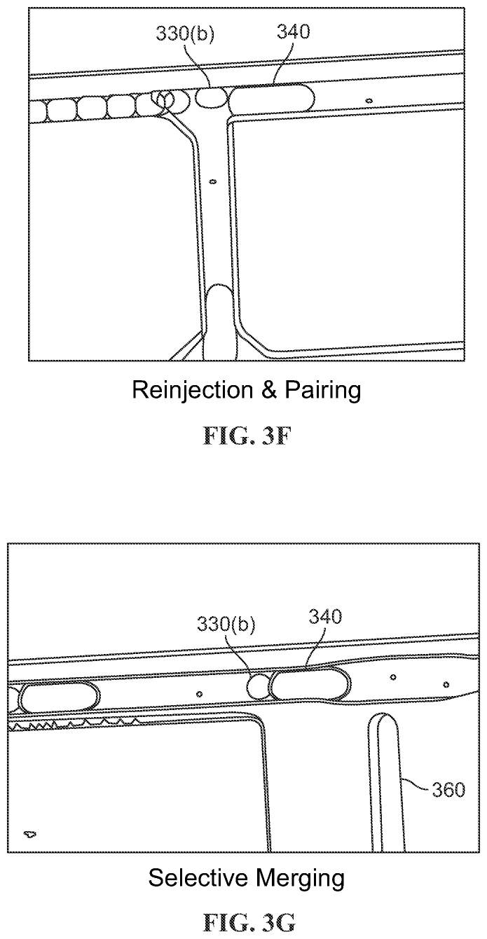

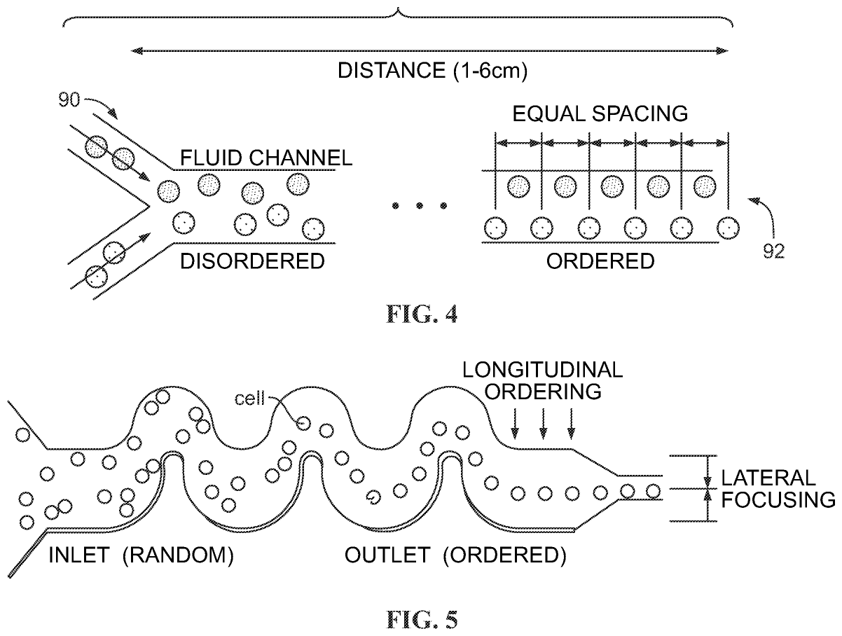

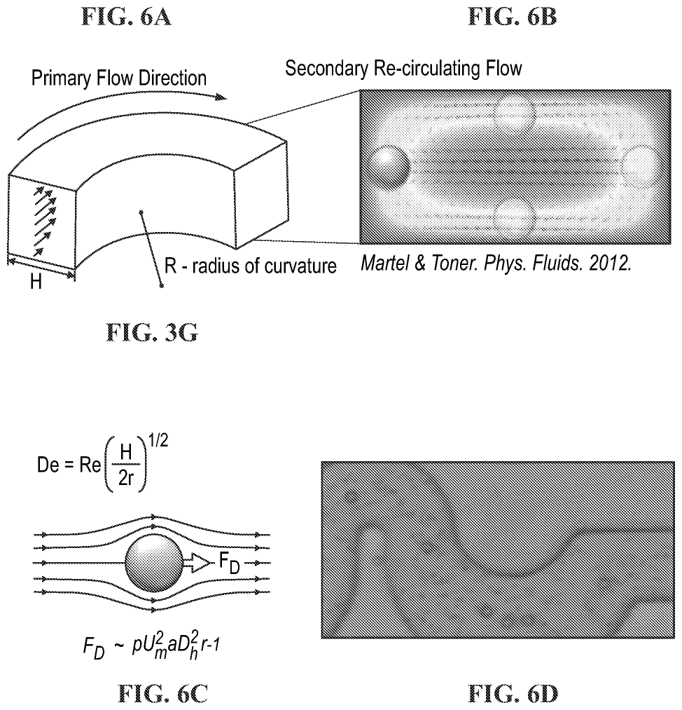

Dual barcoded droplets that encapsulate a unique combination of fluorescent barcode molecules and oligonucleotide barcodes (e.g., optical/oligonucleotide barcodes) are generated in the dual barcoded droplet preparation module, which is described in further detail in the section entitled “Dual Barcoded Droplet Preparation Module” and is illustrated in FIG. 2A and FIG. 3A.

Droplets encapsulating two targets to be tested for an interaction (also referred to as “target droplets”), e.g., a T-cell and an APC, as well as an interaction signaling reporter molecule to signal a target interaction, such as a calcium signaling reporter for detecting T-cell activation, are generated in the inertially ordered cell encapsulation module, which is described in further detail in the section entitled “Inertially Ordered Cell Encapsulation Module” and in FIG. 2B, FIGS. 3A-3B, FIG. 4, FIG. 5, and FIGS. 6A-6D.

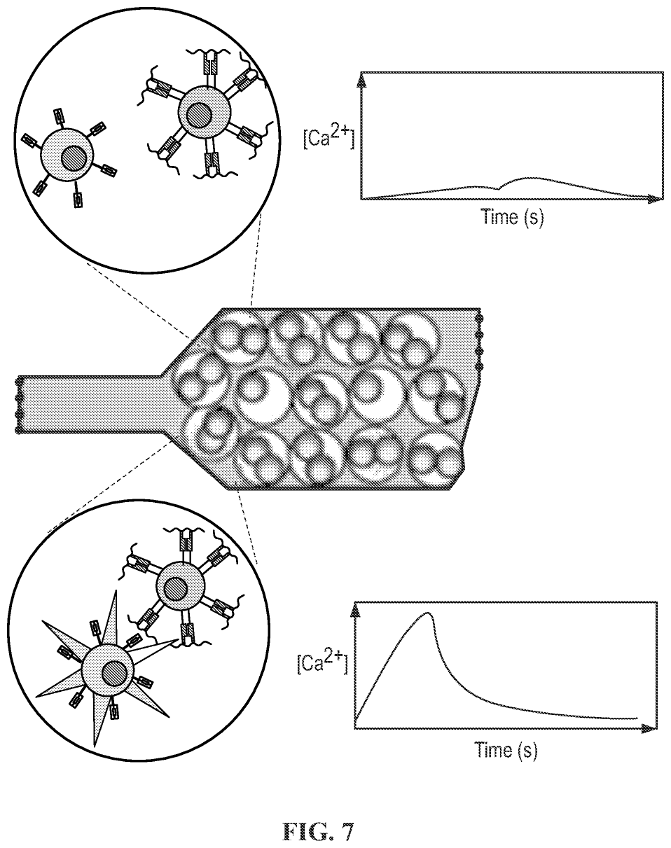

Next, droplets encapsulating the targets, e.g., cells, and the interaction signaling reporter (e.g., calcium signaling reporter) are incubated and the activation profile is recorded (e.g., calcium signaling over time) in the incubation and activation profile module, which is described in further detail in the section entitled “Incubation and Activation Profiling Module” and in FIG. 2C and FIG. 3A, FIGS. 3C-3E, and FIG. 7.

Following incubation, droplets encapsulating particles and the reporter are paired with dual barcoded droplets, and the ratios of two or more, e.g., three, four, five, or more different optical labels, such as fluorescently labeled, optical barcodes are read in the optical barcode detection module, which is described in further detail in the section entitled “Optical Barcode Detection Module” and in FIGS. 2D-2G and FIGS. 3E-3F.

To link the detected optical barcode with T-cell activation, only droplets encapsulating an activated target, e.g., T-cell, are selectively merged with the dual barcoded droplet in the selective droplet merging module, which is described in further detail in the section entitled “Selective Droplet Merging Module” and in FIG. 2E, FIG. 3E, and FIG. 3G.

Accordingly, the TAP-Seq platform described herein enables simultaneous profiling of thousands of cell pairs per second and selective sequencing of only those cell pairs that interact.

A schematic and detail description for the TAP-Seq platform is shown in FIGS. 2A-2G. In brief, dual barcoded (optical/oligonucleotide) droplets are generated (FIG. 2A). T-Two targets to be tested for an interaction, e.g., T-cells and APCs, are co-encapsulated with an interaction signal reporter molecule, e.g., for T-cell activation (FIG. 2B). The target, e.g., cell, pairs, are incubated, and the activation profile is recorded (FIG. 2C). Activated target, e.g., cell, pairs are then selectively merged with the dual barcoded droplet (FIGS. 2D-2E). The optical barcodes are read alongside the activation profile, while the transcriptome is read alongside the oligonucleotide barcodes (FIG. 2F). The activation profiles can be used alongside the RNA sequence for bioinformatic clustering and classification (FIG. 2G).

Once sequencing is completed, all of the transcripts associated with one barcode (or interaction cell pair) can be linked back to the optical barcodes using the embedded UMIs that correlate with the previously recorded optical profile for that droplet. For each color (fluorescence) injected into the droplet, there will be a percentage of that dye that translates to a number of UMI reads for that color. This repeats for each color that was originally placed into the droplet. The unique optical barcode (e.g., 2, 3, 4, or more different colors, each with a unique ratio of the different colors) and UMI percentages will be distinct for each transcriptome and allow for correlation during simple read alignment.

### Dual Barcoded Droplet Preparation Module

Methods and devices described herein involve a dual barcoded (optical barcode and sequence barcode) droplet preparation module for co-encapsulating barcoded beads with a unique combination of fluorescent barcode molecules and oligonucleotide barcodes. The optical barcode molecule allows the droplet to be tracked by optical detection as it moves through the microfluidic device and the sequence barcode allows the droplet to be linked to sequencing reads.

Referring now to FIG. 2A, dual barcoded droplet preparation is described. Each optical barcode 230(a), 230(b), and 230(c) includes an oligonucleotide barcode that includes an Injector ID, which is a unique nucleic acid barcode linked to a unique molecular identifier (UMI), and to an optically detectable, e.g., fluorescent, molecule. The UMI is unique per optical barcode, but each Injector ID is identical per color (or type of optical barcode). So any oligonucleotide barcode molecule in the yellow inlet has the same Injector ID prefix, but a unique UMI. The Injector IDs for Yellow, Red, and Blue are all different. Thus, each droplet is uniquely identified by the ratio of Yellow:Blue:Red Injector ID's (in the case of three colors). The unique UMIs allow you to count the number of Yellow, Red, and Blue Injector ID's and determine that ratio. For a given color, the injector ID is the same in all droplets, but the ratio delivered of each color is unique for each droplet.

Optical barcodes can comprise one or more fluorescent dyes, nanoparticles, microparticles, or any combination thereof. For example, optical barcodes can comprise a plurality of nanoparticles, such as a plurality of quantum dots or Janus particles. In another example, optical barcodes can comprise a plurality of fluorescent dyes, such as between 2-10 fluorescent dyes. Optical barcodes can confer optical properties to oligonucleotide barcodes. Such optical properties can be selected from the group consisting of, for example, an absorbance, a birefringence, a color, a fluorescence characteristic, a luminosity, a photosensitivity, a reflectivity, a refractive index, a scattering, or a transmittance of the particle or a component thereof. The optical barcodes are added to the droplets in specific percentages or ratios, such that the specific percentage of the different colored barcodes provides a unique identifier for each droplet, because no two droplets have the same ratio of the two, three, or more different optical barcodes.

As noted, different controllable amounts of each optical barcode enter into the flow stream from a different channel for each type (e.g., each color) of optical barcode and different amounts of each sequence barcode are injected into a droplet 240 comprising a bead. The droplet 240 is merged with the droplet including the two, three, or more optical barcodes, in different ratios for each droplet, to produce a larger droplet comprising both types of barcodes to provide a dual barcoded droplet 250.

Optical barcodes typically include a UMI, and typically each optical barcode includes a different UMI. For example, in FIG. 2A, optical barcodes 230(a), 230(b), and 230(c) include different UMIs. Accordingly, a droplet shown in FIG. 2A can include three different optical barcodes that include three different UMIs. FIG. 2A includes three optical barcodes, however, any number of optical barcodes (each including a different UMI) suitable for detecting an interaction between two particles can be used in methods and devices described herein. For example, dual barcoded droplets can include any number of optical barcodes, e.g., 1, 2, 3, 4, 5, 6, 7, 8, or more optical barcodes, in a unique percentage for each droplet. Sequence barcodes can include one or more UMIs per droplet, e.g., 1, 2, 3, or more UMIs, that together with the optical barcodes in different percentages or ratios provide the dual barcoded droplets, wherein each droplet is uniquely identified.

Unique barcode concentrations are prepared by varying the relative flow rates of each orthogonal barcode channel. Channels or reservoirs containing a unique optical and DNA barcode are connected to a microfluidic inlet and pressurized by a pressure source, e.g., a Fluigent Flow-EZ® pressure source. Custom software controls the ratio of flow rates from each channel such that the total flow rate is fixed, but the ratio of each barcode channel is varied so that each droplet gets a unique ratio of the, for example, three different types (colors) of optical barcodes. In one embodiment, by prescribing a sinusoidal wave with a defined period and frequency, the full space of barcode combinations can be utilized.

For example, one droplet may contain 40 units of a red fluorescent optical barcode, 15 units of a blue fluorescent barcode, and 20 units of a yellow fluorescent barcode (see FIG. 2D). No other droplets generated in a single analysis run would have this same ratio of the different types/colors of optical barcodes. Another droplet in this analysis might have 15 units of red, 50 units of blue, and 5 units of yellow (see FIG. 2F). Thus, each droplet is uniquely ID by the ratio of Yellow:Blue:Red Injector ID's. The UMIs allow you to count the number of Yellow, Red, and Blue injector ID's and determine that ratio. For a given color, the injector ID is the same in all droplets, but ratio delivered of each color is unique.

Although FIG. 2A depicts encapsulation or injection of sequence barcodes into droplets comprising beads, and then merging that bead into a droplet containing the optical barcodes in different percentages to form a large dual barcoded droplet, methods and devices described herein also encompass encapsulation or injection of dual barcodes into droplets that also include interacting particles. For example, as shown in FIG. 3A, particles 320(a), e.g., cells, such as T-cells, are co-encapsulated with particles 320(b), e.g., cells, such as APCs, and optical barcodes 310(a), 310(b), and 310(c) into droplet 330. These droplets also include an interaction signal reporter molecule. An example of a droplet including interacting particles is labeled as 330(a) and an example of a droplet including non-interacting particles is labeled as 330(b).

Any method known in the art or described herein can be used to prepare dual barcoded droplets for use in methods and devices described herein. Non-limiting examples of dual barcoded droplet preparation techniques that can be used as provided herein are provided in U.S. Application No. US20170009274A1, the relevant disclosures of which are herein incorporated by reference for the purposes and subject matter referenced herein.

In some examples, a plurality of droplets containing cell lysates is provided. The nucleic acids in the lysates may be barcoded so as to enable their sequencing while allowing for the identification of which nucleic acids originated from which droplet and, thus, from single cells. To accomplish this, barcodes that are unique to each cell may be introduced into the droplets. There are a variety of methods that can be used to accomplish this goal. One such method is to introduce a cell into the droplet, wherein the barcode is expressed in the cell, for example, as a high copy number plasmid. This serves to increase the starting concentration of the barcode so that it can be more easily integrated into the sequences of the cell nucleic acids. A suitable plasmid may be, e.g., from about 1 kb to about 3 kb in size.

In some examples, barcodes can be introduced on to the surface of a bead in a droplet, e.g., the surface of a solid polymer bead or a hydrogel bead. Beads for use in the methods and devices described herein can be commercially obtained or synthesized using a variety of techniques. For example, using a mix-split technique, beads with many copies of the same, random barcode sequence can be synthesized.

The unique molecular identifiers (UMIs) can be added to the molecules on the bead surfaces by, for example, a PCR hybridization and extension with primers that have a random UMI sequence. This would permit every individual barcode on a given bead's surface to have a unique identifier, so that bias in the rates at which different molecules are amplified during generation of a sequencing library can be partly corrected by disregarding and/or aggregating duplicated UMIs in quantitation. The UMIs can be used to prepare the optical barcodes by combining the UMIs with different fluorescent molecules, e.g., 2, 3, 4, or more different fluorescent colors for a given analysis run.

With a hard bead, like a polystyrene bead, most of the oligo synthesis will be confined to the surface of the bead. However, hydrogel beads (e.g., polyacrylamide, agarose, or alginate) can also be used, with the advantage that they are porous, permitting the oligos to be synthesized even within the bulk of the beads. These porous beads have the benefit of permitting a much larger number of oligos to be synthesized on/and or in the bead, which may be advantageous for applications that require large numbers of target molecules to be labeled with the barcodes or to control the stoichiometry of the barcode concentration in the subsequent reactions.

### Inertially Ordered Cell Encapsulation Module

Methods and devices described herein involve an inertially ordered cell encapsulation module for co-encapsulating particles within liquid droplets, including formation of picoliter-size monodisperse droplets containing the particles, and inertially ordering the droplets using inertial focusing of the droplets into one of more streamlines within a microfluidic channel. By ordering the particles in a fluid stream within a microfluidic channel before droplet formation, droplets containing specific pairings of target particles can be formed. The target particles can be living cells or other material derived from a biological fluid sample, such as blood, or synthetic materials, such as polymeric beads. For example, fluid droplets containing target particles (e.g., cells such as a T-cell and an APC) can be repeatedly generated in an aqueous fluid (e.g., a saline solution).

As shown in FIG. 2B, the target particles of a first type 210(a) (e.g., T-cells) can be introduced into the channel through a first input branch (the upper branch), while a second target particle type 210(b) (e.g., APCs) can be introduced into the channel through a second separate channel branch, the lower input branch as shown in FIG. 2B. The two types of target particles move from separate input branches into a single channel and are ordered and focused into two streams corresponding to two equilibrium positions on opposite sides of the channel. The uniform spacing in the direction of flow leads to the formation of paired particle droplets 220 when the two lateral flows of oil pull droplets from the aqueous stream with the same (or higher) frequency that particles reach a micro-droplet generator.

In the inertially ordered cell encapsulation module, particles (e.g., solid analyte particles, or cells) are rapidly passed through a high aspect-ratio microchannel until inertially focused into one or more streamlines within the channel, and are flowed into a droplet generator at a defined longitudinal spacing and in an order (e.g., if there are two different types of particles, they are spaced into an alternating order), to result in the formation of a desirable high fraction of liquid droplets with a desired number of particles per droplet (e.g., a single particle per droplet, two particles per droplet, three particles per droplet). For example, the microscope image in FIG. 3B shows inertial ordering and encapsulation of particle 320(a) such that individual droplets include a single particle 320(a). The droplet marked with a white arrow in FIG. 3B is empty.

In general, the microchannel dimensions and fluid flow rate can be selected to provide a fluid stream of ordered particles substantially evenly spaced longitudinally along the length of a portion of the microchannel before entering the droplet generator. In addition, particles, e.g., cells, tend to enter the droplet generator with the frequency of droplet formation. In the resulting droplets, the fraction of multiple-particle liquid droplets is higher than the corresponding fraction of multiple-particle liquid droplets predicted by Poisson statistics.

Non-limiting examples of techniques for inertially ordering droplets including exemplary microchannel dimensions and fluid flow rates are discussed in detail in U.S. Pat. No. 10,174,305 entitled, “Microfluidic Droplet Encapsulation,” which is incorporated herein by reference in its entirety.

Samples can be diluted or concentrated to attain a predetermined ratio before and/or during introduction of the sample into the microfluidic device. In general, the particle to volume ratio can be less than about 50%. In other embodiments, particle to volume ratios can be less than about 40%, 30%, 20%, 10%, 8%, or 6%. More particularly, in some embodiments, particle to volume ratios can be in a range of about 0.001% to about 5%, e.g., in a range of about 0.01% to about 4%. The ratio can also be in the range of about 0.1% to about 3%, e.g., in the range of about 0.5% to about 2%. In general, a maximum particle to volume ratio for a specified particle size and channel geometry can be determined using the formula:

$$Max Volume Fraction = \frac{2 N π {a}^{2}}{3 h w}$$

where N is the number of focusing positions in a channel (e.g., 1, 2, 3, and 4), a is the focused particle diameter, h is the channel height, and w is the channel width. The focusing position refers to a volume where the equilibrium positions of flowing particles converge within a channel. A fluid sample can be diluted or concentrated in batches before introduction into the channel such that the sample ultimately introduced into the system has the required ratio before being introduced to the channel.

Particles suspended within a sample can have any size that allows them to be ordered and focused within the microfluidic channels described herein. For example, particles can have a hydrodynamic size that is in the range of about 40 microns to about 0.01 microns. For example, particles can have a hydrodynamic size that is in the range of about 20 microns to about 0.1 microns; particles can also have a hydrodynamic size that is in the range of about microns to about 1 micron.

Various microfluidic systems and channel geometries can result in longitudinally ordered particles in the direction of flow. Microchannel configurations for ordering a plurality of particles in a fluid stream passing through the microchannel can be designed based on certain parameters relating to the particle size and the microchannel dimensions, including the channel Reynolds number (Rc), the particle Reynolds number (Rp), the Reynolds number based on mean channel velocity (Re), the particle hydraulic diameter (Dh) and the Dean Number (De).

The channel Reynolds number (Rc) describes the unperturbed channel flow: Rc=(UmDh)/v. The particle Reynolds number (Rp) includes parameters describing both the particle and the channel through which it is translating: Rp=R_{c}(a²/Dₕ²)=(Uₘa²)/vD. Both dimensionless groups depend on the maximum channel velocity, Uₘ, the kinematic viscosity of the fluid, and v=μ/ρ (μ and ρ being the dynamic viscosity and density of the fluid, respectively), and Dₕ, the hydraulic diameter, defined as 2wh/(w+h) (w and h being the width and height of the channel). The particle Reynolds number has an additional dependence on the particle diameter, a.

The definition of Reynolds number based on the mean channel velocity can be related to Rc by Rₑ=⅔R_{c}. Channels with curvature create additional drag forces on particles. When introducing curvature into rectangular channels, secondary flows develop perpendicular to the stream direction due to the non-uniform inertia of the fluid. Two dimensionless numbers can be written to characterize this flow, the Dean number (De) based on the maximum velocity in the channel, and the curvature ratio (δ). The Dean number, De=Rc(Dₕ/2r)^{1/2} and the curvature ratio, δ=Dₕ/2r, where r is the average radius of curvature of the channel.

FIG. 4 illustrates cell ordering in a rectangular, straight channel. The separation, ordering, and focusing of particles can occur within straight channels given the appropriate channel dimensions and flow rates. In general, at low flow rates, particles flowing within these exemplary channels distribute uniformly along the length of a channel having (1) a cross-sectional aspect ratio (i.e., ratio of length to width or vice versa) of about 1.5 to 8.0 (preferably about 1.5 to 4.0), and (2) a minimum cross-sectional dimension that is up to about times (e.g., 2.5-10 times) the maximum cross-sectional dimension of a particle passing through the channel in a fluid. In the illustrated embodiment, particles 9 μm in diameter suspended in water were observed in 50 μm-wide square channels, providing a particle diameter to channel diameter ratio of 0.18. An inlet region is shown where the particles are initially uniformly distributed within the fluid but start to focus shortly thereafter to the four channel faces. The degree of focusing increases with Rp (particle Reynolds number) at a given distance along the channel and also increases with the distance traveled along the channel. Preferably the Rp is about 1 or greater. For Rp=2.9 (channel Reynolds number (Rc)=90), complete focusing is observed after a distance of about 1 to 6 cm.

As shown in FIG. 4, the conditions known in the art or described herein with respect to inertial ordering are applied to particles of two different predetermined particle types. The particles 92 of a first type (illustrated as open circles) can be introduced into the channel through a first input branch (the lower branch), while a second particle type (illustrated as closed, shaded circles) can be introduced into the channel through a second separate channel branch, the upper input branch as shown in FIG. 4. As shown, the two types of particles move from separate input branches into a single channel and are initially disordered and then are ordered and focused into two streamlines corresponding to two equilibrium positions on opposite sides of the channel by the time they reach about 1 to 6 cm into the channel. Where the first and second particles are differing cell types, particles having differing chemistries, or some combination thereof, having the particles focused and ordered such that the particles generally alternate between particles of the first type and particles of the second type as they travel down the channel enables greater opportunities to observe and manipulate interactions between particles of the first and second types.

While the illustrated geometry for achieving the effects described with respect to FIG. 4 has an aspect ratio of 1 to 1, similar fluid particle self-effects may be observed with other aspect ratios. In addition to ratios of about 1 to 2, a reduction in symmetry can be observed in rectangular channels having dimensional ratios of approximately 15 to 50, 3 to 5, and 4 to 5. Accordingly, the fluid particle self-ordering effects can be seen for a dimensional aspect ratio of approximately 0.3 (15/50) to a dimensional aspect ratio of approximately 0.8 (4/5), and that the effects can be seen regardless of whether the longer dimension is the width or the height.

Particles in a sample can also be ordered by passing the fluid sample through one or more symmetrically or asymmetrically curved portions of a microfluidic channel. In general, as Rc increases between 0.5 and 5, focusing into two streams of particles in the fluid can occur. As Rc increases, mixed streams are again observed, in agreement with an increased contribution from Dean drag. FIG. 5 shows the separation, ordering, and focusing of particles in a fluid passing through a serpentine asymmetrically curved microfluidic channel. An aspect ratio of a serpentine channel can be substantially 1 to 1 and/or can vary along a length thereof (e.g., the aspect ratio of a serpentine channel can vary over the length of the channel between 1 to 1 and 2 to 1). Particles are randomly distributed in the fluid at the inlet of the channel. As Rc increases between 0.5 and 5, focusing into two streams of particles can occur. As Rc increases, mixed streams are again observed, in agreement with an increased contribution from Dean drag.

The microchannel can also have one or more symmetric curves, but in asymmetric curved channels, the net force generally acts in one direction, biasing a single stable position of the initial distribution, and creating a single focused stream of particles. A time-averaged unidirectional centrifugal and/or drag force favors focusing down to a single stream between Re=1-15 focusing becomes more complex as De increases. Particles are focused to one position of minimum potential with the addition of centrifugal forces or drag forces in the negative x-direction. Complete focusing can also occur for much smaller Rp of about 0.15 and for shorter traveled distances (about 3 mm) than in the case of straight rectangular channels.

Particles in a sample can also be ordered by passing the fluid sample through one or more symmetrically curved portions of a microfluidic channel. In general, as the channel Reynolds number (Rc) increases between 0.5 and 5, focusing into two streams of particles in the fluid can occur. As Rc increases, mixed streams are again observed, in agreement with an increased contribution from Dean drag. An aspect ratio of a serpentine channel can be substantially 1 to 1 and/or can vary along a length thereof (e.g., the aspect ratio of a serpentine channel can vary over the length of the channel between 1 to 1 and 2 to 1). Particles are randomly distributed in the fluid at the inlet of the channel. As Rc increases between 0.5 and 5, focusing into two streams of particles can occur. As Rc increases, mixed streams are again observed, in agreement with an increased contribution from Dean drag.

FIGS. 6A-6D provide a brief overview of the structures and forces involved in inertial ordering in the cell encapsulation module. For example, the microfluidic flow channel can include multiple curved portions. FIG. 6A shows one section of one such curved portion and FIG. 6D shows a microscope image of one section of a curved microfluidic channel with ordered cells passing through. A model of the internal fluid flow through the curved portion of the channel in FIG. 6A is shown in FIG. 6B, which is taken from Martel and Toner, Phys. Fluids, (2012) 24(3):32001. A fluid mechanics equation and diagram showing the forces on a single cell (or other spherical particle) passing through the inertial cell ordering channel of FIG. 6A is shown in FIG. 6C, which is taken from Martel and Toner, Annu. Rev. Biomed. Eng., (2014) 16:371-96.

It should be understood that inertial focusing can be achieved with various types of channels including, but not limited to, curved channels, spiral channels, serpentine channels, and zig zag channels. Non-limiting examples of channel configurations for inertially ordering droplets are discussed in detail in U.S. Pat. No. 10,174,305 entitled, “Microfluidic Droplet Encapsulation,” which is incorporated herein by reference in its entirety.

Various methods can be used for identifying ordered and focused particles within a channel. Labels or tags for identifying or manipulating particles to be focused within the channels can be introduced into the sample before, during, and/or after introduction of the sample into the system. Labeling or tagging of particles is well known in the art for use, for example, in fluorescence-activated cell sorting (FACS) and magnetic-activated cell sorting (MACS), and any of the various methods of labeling can be used. Examples of labeling methods and techniques are discussed in detail in U.S. Pat. No. 6,540,896 entitled, “Microfabricated Cell Sorter for Chemical and Biological Materials” filed May 21, 1999; U.S. Pat. No. 5,968,820 entitled, “Method for Magnetically Separating Cells into Fractionated Flow Streams” filed Feb. 26, 1997; and U.S. Pat. No. 6,767,706 entitled, “Integrated Active Flux Microfluidic Devices and Methods” filed Jun. 5, 2001; all of which are incorporated by reference in their entireties.

### Incubation and Activation Profiling Module

Methods and devices described herein involve detecting interactions between two or more particles by encapsulating the target particles in a droplet with a reporter for detecting interaction between such particles. Any target particles that can interact can be analyzed using methods and devices described herein including, but not limited to, cells, proteins, nucleic acids, drugs, and pathogens such as viruses. Target particles can be analyzed for interaction with particles of the same type (e.g., cell-cell interactions) or particles of a different type (cell-drug interactions).

When analyzing T-cell interactions, two-cell pairs are encapsulated in a single droplet with a fluorescent reporter to measure calcium mobilization, a hallmark of T-cell activation. Each droplet has a different cell pair. As shown in FIG. 2C, droplets comprising the two-cell pairs and the reporter are incubated on a chip and T-cell activation is recorded as calcium signaling over time. Droplet 220(a) includes an interacting pair, which produces a measurable increase in calcium signaling shown in the graph. Droplet 220(b) includes a two-cell pair that does not interact, and therefore no detectable increase in calcium signaling is observed (graph not shown). See also FIG. 3C showing a schematic depiction of droplet 330 including an interacting cell pair, and FIG. 3D showing a microscope image of droplet 330 that has a unique level of fluorescently labeled barcodes (optical barcodes) to distinguish droplet 330 from other droplets in the group. As shown in FIG. 3D, droplet 330 has been activated as signaled by a separate fluorescent emission.

FIG. 7 shows the processing of patient derived TCR-antigen pairs on the TAP-Seq platform. This figure shows the incubation and activation profiling (tracking) module. Following sequencing of a patient's solid biopsy, TCR sequences are identified and transfected into T-cell lines. Similarly, transcripts are analyzed to determine peptides likely to be presented as antigens according to the patient's HLA subtype. Peptide antigens that are likely to be tumor-associated are synthetically presented on APCs cell lines. Both cell lines are co-encapsulated and processed on TAP-Seq. The incubation module is shown with APCs (darker gray) and T-cells (lighter gray). The top left inset shows that when the presented antigen does not correspond to the TCR (Mismatched TCR-Antigen Pair) an immune synapse does not form. There is little to any calcium signaling in these droplets, as indicated in the graph inset in the top right. The lower left inset shows that when the presented antigen does match the TCR (Cognate TCR-Antigen pair) an immune synapse does form. If the immune activation is sufficient, significant calcium dynamics occurs, as shown in the lower right graph inset. This difference in calcium dynamics allows selective sequencing of activated cell pairs.

Various reporter molecules can be used in methods and devices described herein depending on the particles to be analyzed. For example, as described herein, when analyzing T-cell activation, a calcium reporter can be used. In another example, when analyzing drug efficacy, a cell death reporter (e.g., any molecule or dye that crosses the membrane of dead cells but is excluded by viable cells) can be used. In some examples, when analyzing interactions of a cell, the cell can be engineered to express a reporter such as a fluorescent protein.

Target droplets (droplets including particles that might interact) can be incubated any length of time suitable for detecting interaction between the particles in the droplet. For example, droplets can be incubated for about 1 second to about 72 hours, e.g., 5, 10, 50, 75 or 100 seconds or longer, or 1, 2, 5, 10, 15, 20, 25, or 30 minutes or longer, or 1, 2, 5, 10, 15, 20, 24, 30, 50, 48, or 72 hours or longer. In some examples, when shorter incubation times are desired for detecting an interaction, droplets can be incubated in the device. In other examples, when longer incubation times are desired for detecting an interaction, droplets can be removed from the device, incubated, and then returned to the device for further processing as described herein.

### Optical Barcode Detection Module

Methods and devices described herein involve detecting optical barcodes encapsulated in a droplet. For example, as shown in FIG. 2D, when the optical barcode includes a fluorescent molecule in droplet 250, the optical barcodes can be detected using any technique suitable for fluorescence detection.

An example of a useful optical system for fluorescence detection of optical barcodes includes one or more lasers aligned through dichroic mirrors. In such instances, aligned lasers enter an inverted microscope and are focused onto a spot on the microfluidic device using the objective. Fluorescent signals emitted by droplets are collected by the objective and diverted through filters to photomultiplier tubes (PMTs). Signals from the PMTs are processed, which detects droplet fluorescence and triggers a high-voltage power amplifier to generate an on-chip dielectrophoretic force via an electrode.

Each droplet is scanned to detect the specific percentage of the different colored barcodes, thereby identifying each droplet based on its unique color signature. In some examples, optical barcodes are detected using standard high content imaging microscopes for rapid imaging.

Methods and devices described herein include optical barcode detection in droplets containing optical barcodes with or without sequence barcodes. Optical barcode detection can be performed on droplets including interacting particles. Alternatively, droplets including optical barcodes can be scanned and then merged with droplets including interacting particles as described below for the next module.

### Selective Droplet Merging Module

Methods and devices described herein involve selectively merging two or more droplets. Any method known in the art or described herein can be used to merge droplets. For example, as shown in FIGS. 2D-2E, an electrode 270 can be used to merge droplet 220(a) comprising the interacting pair with an interaction reporter, with droplet 250 comprising dual barcodes (optical barcodes and sequence barcode) to form a larger droplet 260 that contains the target particles that have interacted, along with the unique dual barcodes.

In such instances, activation of the electrode is coupled to detection of an activation signal such that droplets with interacting particles are merged to droplets with dual barcodes and droplets with non-interacting particles are not merged, and can be flushed out of the system.

In some examples, droplets comprising the interacting particles are merged with droplets comprising optical barcodes, and then the resulting droplet is merged with a droplet comprising a sequence barcode.

In some examples, droplet merger can be achieved by introducing different droplet types into a microfluidic device from separate inlets in such a way that the droplets flow into a single, joined channel. The droplets can be induced to flow as groups of the different droplet types. This can be accomplished, for example, by joining the outlets of the channels from which the different types are introduced into a single channel, such that the flow of one droplet partly impedes the flow of the droplet in an adjacent channel. After the first droplet enters into the joined channel, the second droplet is able to flow in after it, causing the droplets to be injected into the joined channel as an alternating stream. This concept can be extended to larger numbers of droplets, such as three or more droplets. The droplets can also be induced to flow as groups by making the different droplet types different sizes, which causes the smaller droplets to “catch up” to the larger droplets and naturally form groups. They can then be merged by applying an electric field. Alternatively, the pairs of droplets can be flowed alongside another droplet, such as a larger droplet, and merged with it. They can also be merged with a stream, such as a liquid jet which can then be induced to break into smaller droplets, if desired.

FIG. 3E is a schematic depiction of droplet merger by introducing different droplet types into a microfluidic device from separate inlets in such a way that the droplets flow into a single, joined channel. As shown in FIG. 3E, droplet 340 enters the main microfluidic channel via a side channel and droplet 330(b) “catches up” to droplet 340 to form paired (adjacent) droplets. A microscope image of droplet 330(b) paired with droplet 340 is shown in FIG. 3F. Next, as shown in FIG. 3E, the optical barcodes in droplet 330(b) are read and recorded, and droplet 330(b) is merged with droplet 340 to form droplet 350 using electrode 360, e.g., because cell activation was indicated in droplet 330(b). A microscope image of droplet 330(b) prior to merger with droplet 340 is shown in FIG. 3G.

Accordingly, in some embodiments, methods described herein include merging two or more droplets, wherein the method includes: (a) introducing two or more populations of droplets into a flow channel of a microfluidic device, (i) wherein the flow channel includes a droplet merger section associated with one or more electrodes or one or more portions of one or more electrodes configured to apply an electric field in the droplet merger section of the flow channel, (ii) wherein the two or more populations of droplets are introduced into the flow channel at a single junction from two or more separate inlet channels, respectively, and (iii) wherein the two or more populations of droplets are introduced into the flow channel such that the droplet inputs from each inlet channel at least partially synchronize due to hydrodynamic effects, resulting in the ejection of spaced groups of droplets, in which at least some of the spaced groups of droplets include a droplet from each of the two or more populations of droplets; (b) flowing the spaced groups of droplets into the droplet merger section; and (c) merging droplets within a spaced group by applying an electric field in the droplet merger section of the flow channel using the one or more electrodes or the one or more portions of the one or more electrodes, but only for droplets containing cell-pairs in which the interaction signal has been detected.

In some embodiments, methods described herein comprise merging two or more liquids, wherein the method includes: (a) introducing a first liquid into a flow channel of a microfluidic device as a stream at least partially in contact with an immiscible phase liquid; (b) introducing a droplet including a second liquid into the flow channel; (c) merging the droplet into the stream, thereby combining the first and second liquids; and (d) inducing the stream including the combined first and second liquids to break into individual droplets including the combined first and second liquids.

In some embodiments, the flow channel can include a droplet merger section associated with one or more electrodes or one or more portions of one or more electrodes configured to apply an electric field in the droplet merger section of the flow channel, and the method includes applying the electric filed in the droplet merger section of the flow channel to merge the droplet into the stream.

In some embodiments, the first liquid is introduced into the flow channel under dripping conditions. In other embodiments, the first liquid is introduced into the flow channel under jetting conditions.

Non-limiting examples of droplet merger techniques that can be used in methods and devices described herein are provided in U.S. Patent Application Publication No. US2017/0009274A1, the relevant disclosure of which is herein incorporated by reference for the purposes and subject matter referenced herein.

### Sorting

Methods and devices described herein include sorting of droplets to selectively enrich droplets that include interacting target particles and/or barcodes. Sorting approaches of interest include, by are not necessarily limited to, approaches that involve the use of one or more sorters, e.g., sorters of a microfluidic device, which employ microfluidic valves, membrane valves, bifurcating channels, surface acoustic waves, and/or dielectrophoresis. Sorting approaches which can be utilized in connection with the disclosed methods and devices also include those described by Agresti, et al., PNAS vol. 107, no 9, 4004-4009; and those described in PCT Publication No. WO 2014/028378, the disclosure of each of which is incorporated by reference herein in its entirety and for all purposes. A population, e.g., a population of droplets, can be enriched by sorting, in that a population containing a mix of members having or not having a desired property may be enriched by removing those members that do not have the desired property, thereby producing an enriched population having the desired property.

In some examples, methods described herein include scanning, e.g., optically scanning one or more droplets to facilitate their sorting. As such, in some embodiments, microfluidic devices or portions thereof, e.g., sorters, include one or more detectors, e.g., optical scanners. A variety of suitable optical scanners are known in the art. Such optical scanners may include, e.g., one or more optical fibers for applying excitation energy to one or more discrete entities. In some embodiments, a suitable optical scanner utilizes a laser light source directed into the back of an objective, and focused onto a microfluidic channel through which droplets flow, e.g., to excite fluorescent dyes within one or more discrete entities. Scanning one more discrete entities may allow one or more properties, e.g., size, shape, composition, of the scanned entities to be determined. Sorting may, in turn, be carried out based on the one or more properties. For example, sorting may be based on results obtained from an optical scan of one or more discrete entities.

Properties of droplets which may be detected and by which sorting may be based include, but are not limited to, the size, viscosity, mass, buoyancy, surface tension, electrical conductivity, charge, magnetism, and/or presence or absence of one or more components, e.g., one or more detectable labels (e.g., one or more fluorescent labels), one or more particles. In some embodiments, sorting may be based at least in part upon the presence or absence of one or more cells in the droplet, e.g., one or more detectably labeled cells. In some embodiments, sorting may be based at least in part based upon the detection of the presence or absence of PCR amplification products.

In some examples, sorting includes sorting droplets by one or more of fluorescence-activated cell sorting (FACS), PCR-activated cell sorting (PACS), or magnetic-activated cell sorting (MACS).

Non-limiting examples of droplet sorting techniques that can be used in methods and devices described herein are provided in U.S. Application No. US20170009274A1, the relevant disclosures of which are herein incorporated by reference for the purposes and subject matter referenced herein.

### Fluid Flow

Various techniques exist for moving the sample through a microfluidic channel. For example, a microfluidic system can include a pumping mechanism for introducing and moving the fluid sample into and through one or more microfluidic channels. The pumping mechanism can also regulate and control a flow rate within the channels as needed. A specific pumping mechanism can be provided in a positive pumping configuration, in a negative pumping configuration, or in some combination of both. In one embodiment, a sample can be introduced into the inlet and can be pulled into the system under negative pressure or vacuum using the negative pumping configuration. A negative pumping configuration can allow for processing of a complete volume of sample, without leaving any sample within the channels. Examples of negative pumping mechanisms can include, but are not limited to, syringe pumps, peristaltic pumps, aspirators, and/or vacuum pumps. In other embodiments, a positive pumping configuration can also be employed. A sample can be introduced into the inlet and can be injected or pushed into the system under positive pressure. Examples of positive pumping mechanisms can include, but are not limited to, syringe pumps, peristaltic pumps, pneumatic pumps, displacement pumps, and/or a column of fluid. Oscillations caused by some pumping mechanisms, such as a peristaltic pump, can optionally be damped to allow for proper focusing within the channels.

Flow rates within the channels can be regulated and controlled. For instance, any number and variety of microfluidic valves (micro-valves) can also be included in the system to block or unblock the pressurized flow of particles through the channels. Micro-valves can include one or more mobile diaphragms or flexible membranes formed in a layer above a channel branch, inlet, or outlet such that upon actuation, the membrane is expanded up to decrease resistance within a channel branch, inlet, or outlet, or expanded down into the channel to increase resistance within the same. Further details and discussion of such microfluidic diaphragms are disclosed in PCT Publication No. PCT/US2006/039441 entitled, “Devices and Methods for Cell Manipulation,” filed on Oct. 5, 2007, and incorporated herein by reference in its entirety. Optionally, one or more microfluidic, size-based separation modules or filters can be included to prevent clogging within the channels by preventing certain particle sizes or particle types from entering the channels and/or to facilitate collection of particles for downstream processing.

In general, fluid flow rate can be selected using criteria described herein to achieve the desired result, e.g., a fluid stream of ordered particles substantially evenly spaced along the length of the microchannel before entering the droplet generator, ordering droplets, tracking droplets, pairing droplets, and merging droplets.

For example, when ordering droplets, the flow rate can be 2000-8000 μL/h for the aqueous phase and 3000-9000 μL/h for the oil phase. For example, when tracking droplets in the incubation and activation profiling module, the flow rate can be 10-500 μL/h for the aqueous phase and 100-1000 μL/h for the oil phase. For example, when merging droplets in the selective droplet merging module, the flow rate can be 100-1000 μL/h for the aqueous phase and 500-3000 μL/h for the oil phase. These flow rates enable the generation, detecting, imaging, and monitoring of thousands of droplets per second, e.g., 1000, 2500, 5000, 7500, or even 10,000 droplets per second.

### Droplet Formation

The fluid stream of ordered particles can pass through an outlet of a microfluidic channel through a nozzle (in fluid communication with the outlet of the microfluidic channel) and into a medium suitable to induce droplet formation from the fluid stream. Droplet formation of the fluid can be induced by injecting the fluid into a second immiscible liquid, as described, e.g., by Utada et al, Phys. Rev. Lett. 99, 094502 (2007), incorporated herein by reference in its entirety. The mechanism of droplet formation of the fluid is related to the presence of the surrounding viscous liquid. A liquid forced through an orifice will ultimately break into droplets at slow flows, whereas at faster flows the liquid forms a thin stream that breaks into droplets away from the orifice; these are the dripping and jetting regimes.

The transition between dripping and jetting in a two-phase co-flowing stream. The behavior is characterized by a state diagram that depends on both the capillary number of the outer fluid, Cₒᵤₜ, and the Weber number of the inner fluid, Wᵢₙ; these parameters describe, respectively, the magnitude of the viscous shear forces from the outer liquid and the inertial forces from the inner liquid compared to surface tension forces. A transition from the drop-dripping to jetting behavior is dependent on the capillary number of the outer pinching flow

(Cₒᵤₜ=ηₒᵤₜuₒᵤₜ/γ),
and the Weber number for the inner flow,

(Wᵢₙ=ρᵢₙdᵣᵢₚuᵢₙ2/γ)
where ρ is the density of the fluid, η is the viscosity, γ is the surface tension between the two phases, dₜᵢₚ is the diameter of the forming droplet, and u is the fluid velocity. Both dimensionless numbers should be below O(1) to be certain of stable dripping behavior. Using these parameters, the droplet diameter of the fluid can be calculated based on the composition of the fluid and the immiscible liquid into which the fluid introduced after passing through a nozzle. Droplet formation is affected by parameters including the average velocities of both liquids, their viscosities and densities, surface tension, and the surface chemistry and device geometry, as described by Utada et al, Phys. Rev. Lett. 99, 094502 (2007).

### Uses for the TAP-Seq Platform

One example of a use of the new platform is in calcium signaling studies. Calcium signaling is a critical feature of the immune synapse. The migration dynamics determine the extent of T-cell activation, as well as the downstream effector functions. Existing sequencing platforms are unable to correlate such phenotypic events with the associated transcriptome. With TAP-Seq, one can simultaneously profile calcium mobilization dynamics in activated T-cells and identify the TCR-APC pair via selective single cell sequencing. Droplets are loaded into a microfluidic incubation and activation profiling module. These microfluidic modules incubate droplets while deterministically tracking them. This module is designed to monitor thousands of droplets together for a two-minute incubation period, correlating to the time scale of T-cell activation.

To experimentally verify the incubation and activation profiling module, droplets are generated, with every 100th droplet containing 1 μM fluorescein. A fluorescent microscope is equipped with a fast illumination source (e.g. multiple lasers, white laser, LEDs, pulsed light or traditional wide field illumination), which can be rapidly switched to quickly excite and measure the fluorescent signals inside the droplets. This system allows for both tracking of the optical barcodes in the droplets as well as measuring dynamic changes in fluorescence within the droplets. The droplets continuously move at a controlled rate inside the device, allowing for droplet monitoring by means of fast sweeping of the optical space, or the use of dynamic, ultrafast micro-mirrors (e.g., DLP, DMDs). Machine learning algorithms track droplet during any “resetting” of the imaging position. Alternatively, one can program the system to identify a “triggering” event within a droplet, allowing that one droplet to be recorded at an ultrafast rate to record the full kinetics of the fluorescent changes. Fluorescent signals can be recorded up to 100k to sample the field of view. Each frame is analyzed by custom image analysis software to identify droplet trajectories and record fluorescent profiles.

Depending on the time scales measured, fluorescent signals can be read using photodiodes, PMTs, or more traditional CMOS or CCD sensors. Signals can be read in real time, triggering either data recording, or resetting of the camera position. Image processing software is used to provide this real time field sweep, using pattern recognition, aided by our precise control of the droplets using microfluidics.

The formation of an immune synapse involves the engagement of several interacting signaling proteins in both the APC and the T-cell. However, an artificial immune synapse can be generated via anti-CD3 and anti-CD28 agonist antibody beads. Using Jurkat CD4+ T-cell lines, an in vitro experiment can be performed to identify the minimum dosage of antibody beads necessary to induce activation. Fluo-3 and FuraRed, a ratiometric calcium dye pair used to monitor calcium signaling via fluorescence, are included with the cell and bead solution. Each solution then undergoes bulk RNA sequencing to verify T-cell activation. The minimum activating bead concentration is then tested on the platform. The cells are ordered such that each droplet contains a single Jurkat cell. Each droplet enters the incubation and activation profiling module and is analyzed as described above. The droplets are then collected downstream and sequenced. By comparing the in vitro data with the data from the platform, one can verify that the activation profiles are comparable.

Next, dual barcoded beads are generated and barcoding resolution is quantified. Commercially available beads (e.g., 10× chromium beads) are injected into a microfluidic droplet generator. The bead reinjection is co-flowed with four aqueous solutions (see FIG. 2A), which are delivered at varying ratios using a Fluigent Flow-EZ® pressure source. This system generates unique combinations of the fluorescent and DNA barcodes. Each barcode contains an “Injector ID” sequence followed by a Unique Molecular Identifier (UMI) sequence. The DNA barcode hybridizes to the bead and upon sequencing, reads corresponding to the Injector ID can be identified computationally and enumerated by the number of unique UMI's. The DNA barcode and fluorescent barcode concentrations are correlated and measurement of fluorescent barcodes via microscopy is computationally linked to DNA barcodes via sequencing (FIG. 2F). At the time of dual-barcoded droplet generation, both optical and sequence based barcodes are combined across a range of ratios through precise introduction of different volumes of the corresponding reagents. The ratios of these barcodes thus define a specific dual barcode, which is computationally recorded by appending a tracking file as the software controlling the run proceeds. The ability to resolve the unique optical barcodes via fluorescent microscopy is tested. Then these barcoded droplets are merged with droplets containing a sequencing bead and sequenced (FIG. 2E). The sequencing data determines the minimum resolvable difference in UMI's. The measured optical and bioinformatic resolution is then used to adjust droplet generation parameters so each droplet carries distinct and resolvable barcodes.

FIGS. 2A-2E and FIG. 3A show models of systems for droplet generation and subsequent merging. As consistent barcoding across all samples can be challenging, a pico-injection strategy, where each injector delivers barcodes programmatically, is employed.

Physiologically relevant T-cell activation and immune recognition is only understood via the cell-cell interaction between T-cells and antigen presenting cells (APCs), as classical labelling techniques which aim to capture TCR-antigen interactions (e.g. MHC multimer staining) can fail to detect all relevant interactions. See Rius et al., Peptide-MHC Class I Tetramers Can Fail To Detect Relevant Functional T Cell Clonotypes and Underestimate Antigen-Reactive T Cell Populations. Journal of Immunology (2018), 200(7):2263-2279.

However, existing systems that study interacting cells are low throughput or lack single cell sequencing resolution. Overcoming these limitations, the platform simultaneously profiles T-cell activation and downstream transcriptional changes of interacting cell pairs. Known TCR-peptide antigen pairs can be used to verify the TAP-Seq platform as follows. Following the approach of Gejman et al., Identification of the Targets of T-cell Receptor Therapeutic Agents and Cells by Use of a High-Throughput Genetic Platform, (2020) Cancer Immunol Res 8, 672- 684, a T2 APC cell line exclusively presenting the peptide recognized by the A6 TCR sequence is generated. This APC cell line is co-cultured with a transgenic T-cell line expressing the A6 TCR (co-culture 1) for 24-48 hours to provide sufficient time for immune synapse formation and downstream effector pathways signaling. A control monoculture of A6 T-cells is also maintained. Following incubation, RNA sequencing of both cultures is performed to identify transcriptional changes induced by T-cell activation. The above procedure is then repeated with T2 APCs presenting a non-immunogenic peptide (co-culture 2). These RNA-seq datasets serve as a ground truth for the validation of TAP-Seq as outlined below.

The aforementioned APC cell lines are combined together and processed with the A6 TCR cell line on TAP-Seq. The engineered APC and T-cell lines are encapsulated using the incubation and tracking module previously developed and described above. Each cell is inertially ordered to maximize the number of droplets that contain both cells. Droplets carry either cognate TCR-antigen pairs (resembling co-culture 1) or mismatched TCR-antigen pairs (co-culture 2). Following incubation, cell pairs are merged with barcoded droplets as described earlier. The platform outputs both the calcium mobilization profile indicating extent of immune recognition and the transcriptomes of the APC-T cell pair. Although cell pairs are sequenced together, the individual transcriptomes can be deconvoluted computationally.

As a verification of the platform, RNA-seq data is computationally sorted by highly activated calcium profiles as detected by TAP-Seq. If the detected profile is an accurate measure of immune activation, all sorted profiles correspond to cognate APC-TCR pairs (co-culture 1). These experiments verify the formation of an immune synapse, the classification of activated calcium signaling, and identification of T-cell activation via RNA-sequencing. This validates that the immune synapse is accurately formed and quantifiable with TAP-Seq. This will include identifying strongly activating calcium mobilization profiles and verifying activation with transcriptional changes for activated cells. If the immune synapse takes a significant time to form or the profile itself occurs over a time scale infeasible to incubate microfluidically, creating an incubation and activation profiling module that captures the full calcium mobilization profile might be challenging. In such case, the in vitro experiments are used alongside literature on non-artificial immune synapses to determine the relevant portions of the mobilization profile. The module can then be modified to adjust the delay or incorporate off-device incubation.

In tumor pathology, the presence of tumor infiltrating lymphocytes (TILs) is often a positive prognostic indicator for immune response against tumor associated antigens. Although sequencing methods exist to both identify TCR sequences and potential tumor antigens, there is currently no method to identify the cognate antigen for a given TIL. The TAP-Seq platform is able to identify immunogenic TCR-antigen pairs within a solid biopsy, for example, from head and neck cancer solid biopsies as follows.

Using the commercially available 10× platform, single cell RNA sequencing can be performed on solid biopsies from a select number (for example ten) head and neck cancer patients. This patient cohort undergoes check point inhibitor therapy and solid biopsies will be collected before and after treatment. The RNA sequencing data is then computationally processed to identify TCR sequences from the TILs. Transcripts from the tumor cells are then processed using an antigen presentation algorithm specific to the patient's HLA type. This list of antigens is then compared to healthy tissue data to identify which antigens are possibly tumor associated. Finally, the TCR sequences from the identified TIL and this subset of antigens are transfected into T-cell and APC lines respectively as described above. This process is repeated for each patient and for pre- and post-treatment samples, creating libraries of cells reflecting the TCR and antigen space of these tumors.

Following the establishment of the APC and T-cell libraries for each patient sample, the samples are individually processed using the TAP-Seq platform. Because the probability of cognate TCR-antigen pairing in the droplet is low, thousands of cells for each clone in the APC/T-cell libraries are included. Additionally, the cells are inertially ordered to optimize the number of droplets containing both an APC and T-cell. To ensure sequencing is targeted, the observed calcium dynamics is used to selectively merge only sufficiently interacting cell pairs as shown in FIG. 7. With the TAP-Seq platform, one can identify new APC- TCR pairs that emerge following check point inhibitor therapies on a per patient basis. By considering each patient's clinical progression, the TCRs likely to be productive at targeting the tumor can be identified. By analyzing samples from cancer patients, patient specific TCR- antigen pairs are identified and their functional interaction quantified. Also, by considering clinical progression, TCR-antigen pairs emerging from check point blockade therapy can be identified. Generation of the APC library and the bulk assays mirror previous protocols (38). If the calcium dynamics for TCR-antigen pairs varies considerably from known data, additional incubation experiments can be prior to sequencing to identify the activated calcium profile.

### EXAMPLES

In order that the invention described may be more fully understood, the following examples are set forth. The examples described in this application are offered to illustrate the methods and compositions provided herein and are not to be construed in any way as limiting their scope.

### Example 1: Development of the TAP-Seq Platform for Combined Single Cell Immune Profiling and Sequencing

This Example describes development of TAP-Seq platform that can simultaneously profile calcium mobilization dynamics in activated T-cells and identify the TCR-APC pair via selective single cell sequencing. First, experiments are performed to study and improve the platform's ability to resolve time dependent immune activation dynamics and then maximize its barcode space to enable analysis of large TCR-APC libraries.

Calcium signaling is a critical feature of the immune synapse. The migration dynamics determine the extent of T-cell activation, as well as the downstream effector functions. Existing sequencing platforms are unable to correlate such phenotypic events with the associated transcriptome. To address this technology gap, we developed TAP-Seq, a novel microfluidic platform that can both record optical phenotypes over time and correlate them with the corresponding transcriptome via a dual barcoding approach.

To validate droplet incubation and tracking module of the TAP-Seq platform, droplets are loaded into a microfluidic incubation and activation profiling module. These microfluidic modules incubate droplets while deterministically tracking them. The module is designed to monitor thousands of droplets together for a specified incubation period, e.g., two minutes, correlating to the time scale of T-cell activation.

To experimentally verify the module, droplets are generated with every 100^{th} droplet containing 1 μM fluorescein. A fluorescent microscope is used to sample the field of view at frames per second in both Bright Field and GFP channels. Each frame is analyzed by custom image analysis software to identify droplet trajectories and record fluorescent profiles.

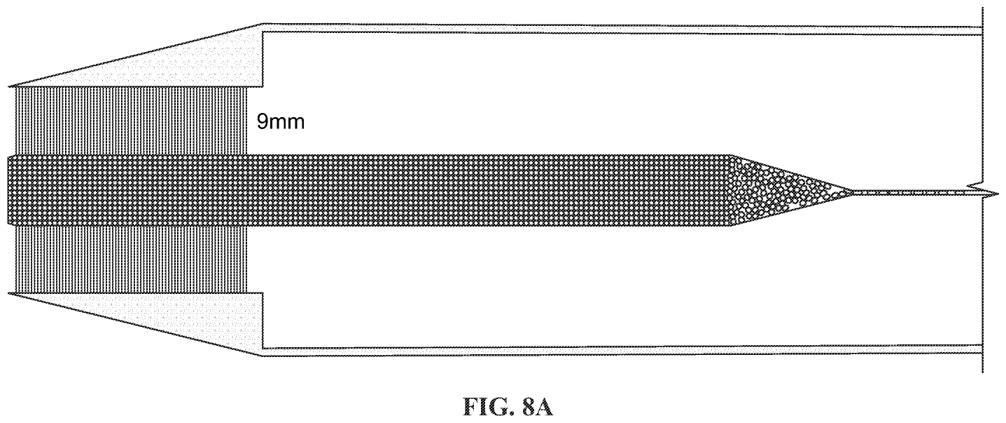

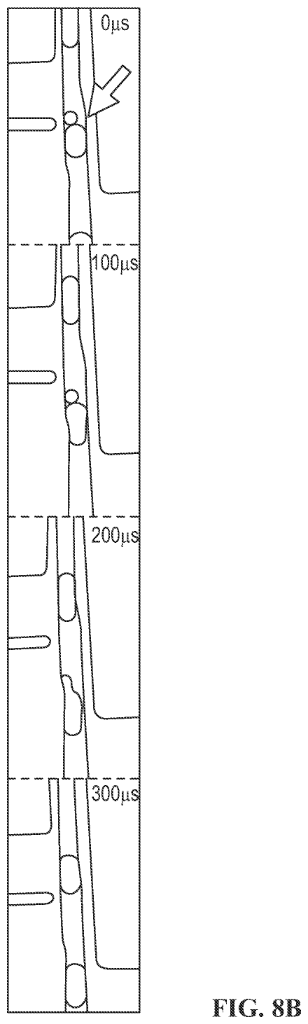

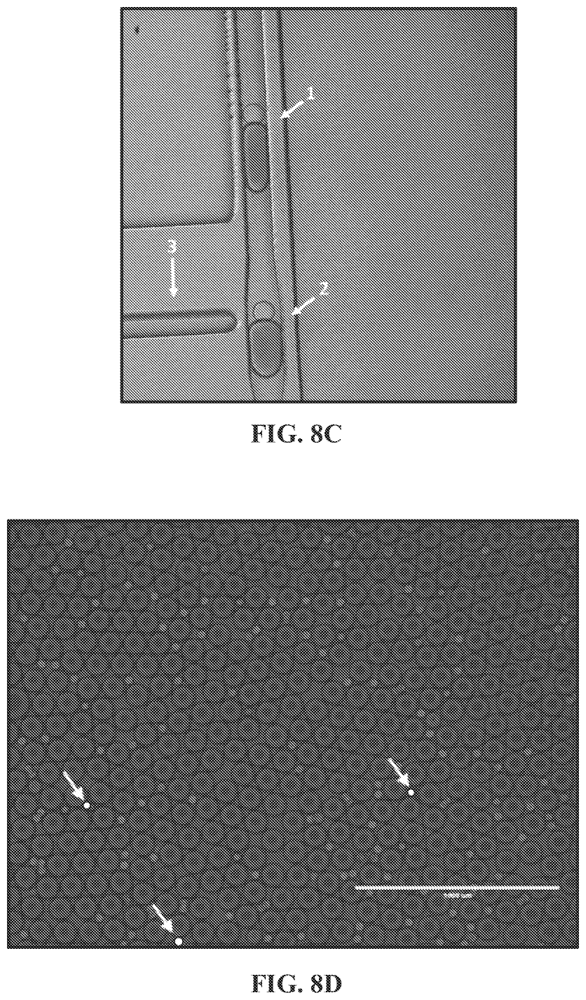

A microscope image of an example of the incubation and activation profiling module is shown in FIG. 8A. The panel on the left shows packing of the droplets in the module, which is achieved by pulling debris and fluid out of the module via microchannels that are perpendicular to the longitudinal axis and direction of flow of the module. The debris and fluid is collected into the triangular-shaped reservoirs and flowed out of the device via waste channels shown parallel to the incubation and activation profiling module at the “top” and “bottom” of the image of the device shown in FIG. 8A.

As the droplets move from along the module shown in FIG. 8A from the portion of the incubation and activation profiling module shown in the left panel to the portion of the module shown in the middle panel, the droplets become more ordered. Hundreds to thousands of ordered droplets shown in the middle panel of FIG. 8A are monitored to determine whether particles inside the droplets interact. The droplets are then funneled into a single stream as shown in the right panel of FIG. 8A for pairing with dual barcoded droplets.

Pairing of the target droplets (droplets including particles that might interact) with the barcoded droplets is shown in FIG. 8B. The target droplet (small droplet) is shown paired with the barcoded droplet (larger droplet) in the 0 μs panel of FIG. 8B. The paired droplets are marked with an arrow. In the 0 μs panel of FIG. 8B, the paired droplets are shown in front of an electrode that is selectively activated if the target droplet includes interacting particles. In this image, the target droplet contains interacting particles and is merged with the barcoded droplet. In FIG. 8B, the 100 μs panel shows the paired droplets, the 200 μs panel shows the paired droplets beginning to merge, and the 300 μs panel shows the droplets merged.

A close up image of one paired droplet (marked by arrow 1) and another paired droplet (marked by arrow 2) are shown traveling in the channel in FIG. 8C. The paired droplet marked by arrow 2 is in front of the electrode (marked by arrow 3) in FIG. 8C. Merged droplets containing fluorescein are marked by arrows in the microscope image shown in FIG. 8D.

The ability to profile T-cell activation and synapse formation is demonstrated as follows. The formation of an immune synapse involves the engagement of several interacting signaling proteins in both the APC and T-cell. However, an artificial immune synapse can be generated via anti-CD3 and anti-CD28 agonist antibody beads. Using Jurkat T-cell lines, an in vitro experiment is performed to identify the minimum dosage of antibody beads necessary to induce activation. Fluo-3 and Fura Red, a ratiometric calcium dye pair used to monitor calcium signaling via fluorescence, is included with the cell and bead solution. Each solution will then undergo bulk RNA sequencing to verify T-cell activation. The minimum activating bead concentration will then be tested on the TAP-Seq platform. Cells are ordered such that each droplet contains a single Jurkat cell using inertial microfluidics. Each droplet enters the incubation and activation profiling module and is analyzed as described above. The droplets are then collected downstream and sequenced. By comparing the in vitro data with the data from the TAP-Seq platform, comparable activation profiles can be verified.

Next, dual barcoded beads are generated and barcoding resolution is quantified. Commercially available beads (inDrop) are injected into a microfluidic droplet generator. The bead reinjection is co-flowed with four aqueous solutions (FIG. 2A), which are delivered at varying ratios using a Fluigent Flow-EZ® pressure source. This method generates unique combinations of the fluorescent and DNA barcodes. Each barcode contains an “Injector ID” sequence followed by a Unique Molecular Identifier (UMI) sequence. The DNA barcode hybridizes to the bead and upon sequencing, reads corresponding to the Injector ID can be identified computationally and enumerated by the number of unique UMI's. Importantly, the DNA and fluorescent barcode concentration are correlated and measurement of fluorescent barcodes via microscopy can be computationally linked to DNA barcodes via sequencing (FIG. 2F). First, the ability to resolve unique optical barcodes via fluorescent microscopy is quantified. Then, these barcoded droplets are merged with droplets containing single Jurkat cells and sequenced (FIG. 2G). This sequencing data determines the minimum resolvable difference in UMI's. The measured optical and bioinformatic resolution is used to adjust our droplet generation parameters so each droplet carries distinct and resolvable barcodes.

Taken together, the TAP-Seq platform is designed, built and validated using surrogate biological models.

### Example 2: Verification of the TAP-Seq Platform with Known TCR-Peptide Antigen Pairs

Physiologically relevant T-cell activation and immune recognition is best understood via the cell-cell interaction between T-cells and antigen presenting cells. However, existing systems that study interacting cells are low throughput or lack single cell sequencing resolution (38,39). To overcome these limitations, the TAP-Seq platform simultaneously profiles T-cell activation and downstream transcriptional changes of interacting cell pairs.

First, APC and T-cell lines with known immunogenicity are established. A T2 APC cell line exclusively presenting the peptide associated with the A6 TCR sequence is generated according to methods described in Gejman et al., Identification of the Targets of T-cell Receptor Therapeutic Agents and Cells by Use of a High-Throughput Genetic Platform, (2020) Cancer Immunol Res 8, 672-684.

This APC cell line is cocultured with a transgenic T-cell line expressing the A6 TCR (coculture 1) for 24-48 hours to provide sufficient time for immune synapse formation (38). A control monoculture of A6 T-cells is also maintained. Following incubation, RNA sequencing of both cultures is performed to identify transcriptional changes induced by T-cell activation. Then, the above procedure is repeated with APCs presenting a non-immunogenic peptide (coculture 2). These RNA-seq datasets serve as a ground truth for the validation of TAP-Seq as described below.

Verifying TAP-Seq is performed by profiling and sequencing TCR-antigen pairs with known immunogenicity. The aforementioned APC cell lines are combine together and processed with the A6 TCR cell line on TAP-Seq. The engineered APC and T-cell lines are encapsulated using the incubation and tracking module developed as described herein. Importantly, each cell is inertially ordered to maximize the number of droplets that contain both cells (FIG. 2B). Droplets carry either cognate TCR-antigen pairs (resembling coculture 1) or mismatched TCR-antigen pairs (coculture 2). Following incubation, cell pairs are merged with barcoded droplets as described herein. The TAP-Seq platform outputs both the calcium mobilization profile indicating extent of immune recognition and the transcriptomes of the APC-T cell pair. Although cell pairs are sequenced together, the individual transcriptomes can be deconvoluted computationally. As a verification of the TAP-Seq platform, RNA-seq data is computationally sorted by highly activated calcium profiles as detected by TAP-Seq. If the detected profile is an accurate measure of immune activation, it is understood that all sorted profiles correspond to cognate APC-TCR pairs (coculture 1). These experiments verify the formation of an immune synapse, the classification of activated calcium signaling, and identification of T-cell activation via RNA-sequencing.

Taken together, the experiments described above validate that the immune synapse is accurately formed and quantifiable with TAP-Seq. These experiments include identifying strongly activating calcium mobilization profiles and verifying activation with transcriptional changes for activated cells.

### Example 3: Identification of Immunogenic Tumor Antigens and the Corresponding TCR from Solid Biopsies

In tumor pathology, the presence of tumor infiltrating lymphocytes (TILs) is often a positive prognostic indicator for immune response against tumor associated antigens. Although sequencing methods exist to both identify TCR sequences and potential tumor antigens, there is currently no method to identify the cognate antigen for a given TIL or to describe any quantifiable parameters of their interaction in cells. TAP-Seq fills a critical role in identifying immunogenic TCR-antigen pairs within a solid biopsy.

TCR and antigen sequences from head and neck cancer solid biopsies are identified as follows. Using the commercially available 10× platform, single cell RNA sequencing is performed on solid biopsies from 10 head and neck cancer patients. This patient cohort will undergo check point inhibitor therapy and solid biopsies are collected before and after treatment. The RNA sequencing data is computationally processed to identify TCR sequences from the TILs. The tumor transcripts are processed using an antigen presentation algorithm specific to the patient's HLA. This list of antigens is then compared to healthy tissue data to identify which antigens are possibly tumor associated. Finally, the TCR sequences from the identified TIL and this subset of antigens are transfected into APC and T-cell lines as described above. This process is repeated for each patient and for pre- and post-treatment samples.

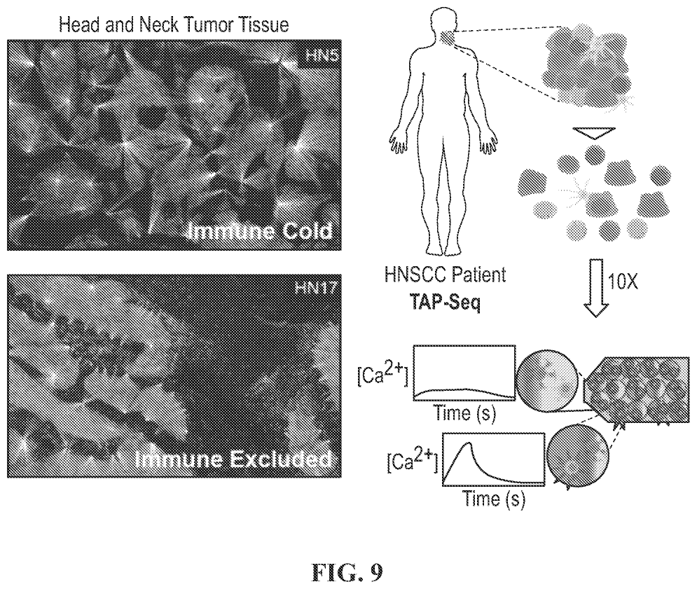

Leveraging TAP-Seq to identify emerging TCR-antigen pairs from checkpoint inhibitor therapy is schematically depicted in FIG. 9 and will be performed as follows. Following the establishment of the APC and T-cell libraries for each patient sample, the TAP-Seq platform will be used to individually process them. Because the probability of cognate TCR-antigen pairing in the droplet is low, thousands of cells for each clone will be included in the APC/T-cell libraries. Additionally, the cells will be inertially ordered to optimize the number of droplets containing both an APC and T-cell. To ensure sequencing is targeted, the observed calcium dynamics is used to selectively merge only sufficiently interacting cell pairs. With the TAP-Seq platform, new APC-TCR pairs that emerge following check point inhibitor therapies on a per patient basis are identified for the first time. By considering each patient's clinical progression, which TCRs are likely to be productive at targeting the tumor can also be identified.

In sum, the TAP-Seq platform will be used to identify patient specific TCR-antigen pairs and quantify their functional interaction by analyzing samples from cancer patients. By considering clinical progression, the TAP-Seq platform can also be used to identify TCR-antigen pairs emerging from check point blockade therapy. These results not only provide the final translational benchmarks for TAP-Seq, they also provide real insights into actionable data for immunotherapy development.

### Example 4: Computer Implemented Methods of Use of the TAP-Seq Platform

Optical barcodes are used to observe droplets containing T-cells and APCs moving through the system under microscopic monitoring. Once an immune synapse is formed and T-cells are activated the dynamics are recorded, one can computationally record the dynamics and the corresponding optical barcode information. One then uses the optical barcodes as a selection marker to enrich for cell pairs of interest by targeted sequencing via selective merging. Following the merging step, transcripts from the T-cell, APC, and the DNA sequence barcodes are all hybridized to the barcoded bead. In the droplet, an RT step is performed and the transcripts and DNA sequence barcodes are extended onto the barcoded bead and therefore the cell barcode/UMI from the barcoded bead is added as a prefix to the transcripts and injected DNA sequence barcode. All DNA is then sent for sequencing, e.g., next generation sequencing, such as sequencing-by-synthesis.

Similar to standard single cell sequencing workflows, the reads are then processed by pulling out unique cell barcodes and reads are quantified by counting UMI's. Reads from distinct droplets can then be discriminated by their unique cell barcode. Therefore, one can cluster transcript reads and injected DNA sequence barcodes together. The UMI's that flank the DNA sequence barcode will be counted to identify the unique injected DNA sequence barcode, and those counts will directly correspond to the intensity of fluorescent optical barcode detected during microscopy.

The methods described above can be computer implemented, for example, using a computer program. In such instances, the program simulates the barcode space by generating 3 sine waves with given periods and phases. The value of these sinewaves at any point in time describes the unique combination of three different types (yellow, red, and blue) optical barcodes. The program shows graphs that indicate the optical barcode space. A graph is shown for each optical barcode. For example, if three optical barcodes are used, then the program shows three graphs including optical barcode 1 vs optical barcode 2, optical barcode 1 vs optical barcode 3, optical barcode 2 vs optical barcode 3. The user can then adjust the period and phase to maximally cover the potential optical barcode combinations. The optical barcode space includes a combinatoric extension described below.

Once the user is satisfied with the optical barcode space, the user can submit the sine waves to the three pressure controllers. The code submits a complex waveform to a fourth pressure source that balances the total flow rate such that droplet production is monodisperse and regular.

Following one run of the sinewaves, the program immediately performs amplitude modulation to extend the optical barcode space. The first pressure source is set to a constant low pressure value and the other pressure sources run the same sinewave with an expanded amplitude range. For example, if the original waves prescribed are: [A*sin(t/p1+phi1)+A,A*sin(t/p2+phi3)+A,A*sin(t/p3+phi3)+A], the first source would be set to A and the second adjusted to: [A,A*sin(t/p2+phi2)+2A,A*sin(t/p3+phi3)+A]. This is repeated for each permutation (e.g., pressure source 1 set to low, pressure source 2 increased by A; pressure source 1 set to low, pressure source 3 increased by A; pressure source 1 set to low, pressure sources 2 and 3 increased by 0.5 A) so that all droplets in a given run are each provided with a unique ratio of the three different types (colors) of optical barcodes. Then the UMI sequence barcode is added to provide a further unique identifier for each droplet.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for analyzing an interaction between two or more particles, the method comprising:
(a) inertially ordering the particles into spaced and ordered streams of particles;
(b) co-encapsulating in individual droplets two or more of the spaced and ordered particles and an activation reporter to form a plurality of target droplets;
(c) co-encapsulating in individual droplets a plurality of different optical barcodes to generate a plurality of barcoded droplets, wherein a specific ratio of the different optical barcodes is used to uniquely identify each of the individual barcoded droplets;
(d) determining interaction between the particles in each target droplet by monitoring each target droplet for a presence or absence of the activation reporter to identify target droplets positive for interacting, activated particles;
(e) merging each identified target droplet with an adjacent barcoded droplet to generate merged droplets; and
(f) sequencing nucleic acids in the merged droplets to determine the sequence of any nucleic acids in the particles and to determine the sequence of any barcodes in the merged droplets;
wherein step (c) further comprises co-encapsulating in the individual barcoded droplets at least one sequence barcode to generate a plurality of dual barcoded droplets and step (e) comprises merging each identified target droplet with an adjacent dual barcoded droplet; or
wherein steps (b) and (c) are combined to co-encapsulate in individual target droplets the two or more particles and the activation reporter as well as the plurality of different optical barcodes to generate the target droplets, and step (e) comprises merging each identified target droplet with a second droplet comprising a sequence barcode to form the merged droplet; or
wherein step (c) further comprises merging the target droplets with the barcoded droplets to generate optically barcoded target droplets, and step (e) comprises merging each identified optically barcoded target droplet with a second droplet comprising a sequence barcode.

2. The method of claim 1, wherein step (c) further comprises co-encapsulating in the individual barcoded droplets at least one sequence barcode to generate a plurality of dual barcoded droplets and step (e) comprises merging each identified target droplet with an adjacent dual barcoded droplet.

3. The method of claim 1, wherein steps (b) and (c) are combined to co-encapsulate in individual target droplets the two or more particles and the activation reporter as well as the plurality of different optical barcodes to generate the target droplets, and step (e) comprises merging each identified target droplet with a second droplet comprising a sequence barcode to form the merged droplet.

4. The method of claim 1, wherein step (c) further comprises merging the target droplets with the barcoded droplets to generate optically barcoded target droplets, and step (e) comprises merging each identified optically barcoded target droplet with a second droplet comprising a sequence barcode.

5. The method of any one of claims 1 to 4, wherein the particles are T-cells and Antigen Presenting Cells from a patient who has cancer, and wherein method is used to analyze TCR-antigen interactions in a sample of a patient's tumor and information about the patient's TCR-antigen interactions is used to select a TCR-based immunotherapy that will recognize the patient's tumor to stimulate an anti-tumor response.

6. The method of any one of claims 1 to 4, wherein the particles are immune cells and diseased cells from a patient who has an autoimmune disease, and wherein the method is used to analyze interactions in a sample between the patient's immune cells and diseased cells to identify immune cells responsible for an unwanted autoimmune response.

7. The method of any one of claims 1 to 4, wherein the particles are tumor cells from a patient and one or more specific drugs taken by the patient whose tumor has developed a resistance to the one or more specific drugs, wherein the method is used to analyze interactions between the patient's cells and the one or more specific drugs to identify genetic mutations responsible for the patient's drug resistance.

8. The method of any one of claims 1 to 4, wherein the particles are a bacteria or a virus that has become resistant to a drug, and the method is used to determine any genetic cause of the drug resistance.

9. The method of any one of claims 1 to 8, wherein inertially ordering the particles comprises flowing the particles through one or more channels at a flow rate that is controlled to induce inertial focusing.

10. The method of claim 9, wherein the one or more channels comprise one or more curved channels having a Dean number of up to about 30.

11. The method of claim 10, wherein the curved channel is symmetrically curved and wherein a channel Reynolds number (Rc) of between about 0.5 and 5.0 causes focusing of particles into two longitudinally ordered streams of particles.

12. The method of claim 10, wherein the curved channel is asymmetrically curved and a channel Reynolds number (Rc) of between about 1.0 and 15.0 causes focusing of particles into a single longitudinally ordered stream of particles.

13. The method of claim 10, wherein the curved channel is asymmetrically curved and a mean channel velocity (Re) is set to about 2.5 to 5.0, wherein Re equals ⅔ of the channel Reynolds number (Rc).

14. The method of claim 10, wherein a Dean number for the curved channel ranges from about 1 to about 20, and wherein a ratio of particle size to hydraulic diameter of the first microchannel is less than about 0.5.

15. The method of any one of claims 1-14, wherein each optical barcode in the plurality of optical barcodes comprises an injector ID nucleic acid sequence, a fluorescent molecule, and a unique molecular identifier (UMI), wherein all injector ID nucleic acid sequences for one fluorescent color are the same, but are different from injector ID nucleic acid sequences for optical barcodes having a different fluorescent color, and wherein all UMIs are different.

16. The method of any one of claims 1-15, wherein the sequence barcode comprises a UMI.

17. The method of any one of claims 1-16, wherein the optical barcodes confer one or more optical properties selected from the group consisting of an absorbance, a birefringence, a color, a fluorescence characteristic, a luminosity, a photosensitivity, a reflectivity, a refractive index, a scattering, or a transmittance of the particle, or a component thereof.

18. The method of any one of claims 1-17, wherein the target droplets and the dual barcoded droplets are merged by applying an electric field that causes destabilization of the droplets such that they are merged together.

19. The method of any one of claims 1-18, wherein the target droplets and the dual barcoded droplets are merged by droplet-stream merger, droplet-jet merger, or both. The method of any one of claims 1-19, wherein the particles are selected from the group consisting of cells, eggs, bacteria, fungi, virus, algae, any prokaryotic or eukaryotic cells, organelles, exosomes, beads, reagents, drugs, small molecules, proteins, antibodies, enzymes, and nucleic acids.

21. The method of claim 20, wherein the particles comprise a T-cell and an antigen presenting cell (APC) and the activation reporter is a calcium activation reporter.

22. The method of any one of claim 1-21, wherein the sequencing comprises single-cell RNA sequencing or single-cell DNA sequencing.

23. The method of any one of claims 1-22, wherein one or more steps are performed in a microfluidic device.

24. The method of any one of claims 1-23, wherein the method is carried out at a flow rate that enables production and monitoring of at least 100, 500, 1000, 2500, 5000, 7500, or droplets per second.

25. A microfluidic system comprising:
(a) a dual barcoded droplet preparation module comprising a plurality of channels for receiving one or more barcodes and a droplet generator comprising a nozzle in fluid communication with the plurality of channels;
(b) an inertially ordered cell encapsulation module comprising
(i) a microchannel having an inlet, an outlet, and a minimum cross-sectional dimension D configured to receive a fluid sample containing multiple particles having a maximum individual cross-sectional dimension of at least 0.1 D, and
(ii) a droplet generator comprising a nozzle in fluid communication with the outlet of the microfluidic channel;
(c) an incubation and activation profiling module comprising an inlet end, a middle section, and on outlet end, wherein the inlet end comprises a central channel for droplets and a plurality of microchannels arranged in fluid communication with and one or both sides of the central channel to allow excess fluid and other waste materials in the fluid sample to flow out of the central channel while maintaining droplets within the central channel, and wherein the outlet end comprises a narrowing channel to allow the droplets to become arranged in single file when exiting the outlet end;
(d) an optical barcode detection module comprising one or more optical detection devices; and
(e) a selective droplet merging module comprising a channel and an electrode configured to apply an electric field in the channel sufficient to cause adjacent droplets to merge into one larger droplet.

26. The microfluidic system of claim 25, further comprising one or more pumping mechanisms in fluid communication with the microfluidic system and arranged to move a fluid sample through the microfluidic system.

27. The microfluidic system of claim 25 or claim 26, wherein the electrode is controlled to apply an electric field when the one or more optical detection devices signal an activated particle.

28. The microfluidic system of any one of claims 25-27, wherein the microchannel comprises a curved microchannel and has a Dean number of up to about 30.

29. The microfluidic system of claim 28, wherein the curved microchannel is symmetrically curved and has a channel Reynolds number (Rc) of between about 0.5 and 5.0 to cause focusing of particles into two longitudinally ordered streams of particles.

30. The microfluidic system of claim 28, wherein the curved microchannel is asymmetrically curved and has a channel Reynolds number (Rc) of between about 1.0 and to cause focusing of particles into a single longitudinally ordered stream of particles.

31. The microfluidic system of claim 28, wherein the curved microchannel is asymmetrically curved and has a mean channel velocity (Re) set to about 2.5 to 5.0, wherein Re equals ⅔ of a channel Reynolds number (Rc).

32. The microfluidic system of claim 28, wherein a Dean number for the curved microchannel ranges from about 1 to about 20, and wherein a ratio of particle size to hydraulic diameter of the first microchannel is less than about 0.5.

33. The microfluidic system of any one of claims 25-32, further comprising one or more controllers comprising hardware or software, or both hardware and software, configured to control one or more of:
(i) the one or more pumping mechanisms to regulate the flow rate of the fluid sample within the microfluidic system,
(ii) the injection of different optical barcodes into the dual barcoded droplet preparation module in precise, unique ratios per droplet,
(iii) the one or more optical detection devices to detect and/or monitor for an activation reporter in any activated target droplets,
(iv) the one or more optical detection devices to detect and/or monitor optical barcode ratios per droplet, and
(iv) the electrode.

34. The microfluidic system of any one of claims 25-33, further comprising one or more conduits arranged to flow a fluid sample containing droplets between the dual barcoded droplet preparation module and the inertially ordered cell encapsulation module; between the inertially ordered cell encapsulation module and the incubation and activation profiling module; between the incubation and activation profiling module and the optical barcode detection module; and/or between the optical barcode detection module and the selective droplet merging module.

35. The microfluidic system of any one of claims 25-34, further comprising a sequencing system.

36. The microfluidic system of claim 35, wherein the sequencing system comprises a sequencing-by-synthesis system.
